(19) United States
(12) Patent Application Publication
(10) Pub. No.: US 2015/0201961 A1
(43) Pub. Date: Jul. 23, 2015

(54) DEVICE AND METHOD FOR PRODUCING A FIRST SKIN INCISION IN SURGICAL PROCEDURES AND MARKING ALONG THE MARGINS OF THE INCISION

(71) Applicant: T-Medical Innovations, Ltd., Haifa (IL)

(72) Inventor: Dror Arnon, Haifa (IL)

(21) Appl. No.: 14/562,807

(22) Filed: Dec. 8, 2014

Related U.S. Application Data
(63) Continuation-in-part of application No. PCT/IL2013/050491, filed on Jun. 7, 2013.
(60) Provisional application No. 61/658,497, filed on Jun. 12, 2012.

(51) Int. Cl. A61B 17/3209

(52) U.S. Cl. CPC A61B 17/32093; A61B 17/3213

(57) ABSTRACT

A method and a device for producing a skin incision in surgical procedures and marking along the margins of the incision are disclosed.

### REFERENCE TO CROSS-RELATED APPLICATION

This application is a continuation-in-part of PCT/IL2013/050491 filed 7 Jun. 2013 and claims the benefit of U.S. Provisional Application No. 61/658,497, filed Jun. 12, 2012.

### FIELD OF THE INVENTION

The present invention relates to a method and a device for producing a skin incision in surgical procedures and marking along the margins of the incision, and more particularly, to a device enabling controlled and balanced performance of a straight and/or precise incision at a desired length and at a desired uniform depth.

### BACKGROUND OF THE INVENTION

The standard stages of performance of open surgical procedures include the performance of a first incision. This is a superficial incision, at a typical depth of few millimeters, into the external layers of the skin, marking the path along which the surgeon will deepen the incision later in the surgical procedure. The first incision can vary in depth, length and shape according to the type of procedure, the body type of the patient, the location of the incision on the patient's body and various other parameters.

The path of the first incision dictates the shape of the scar that will remain on the skin of the patient after the surgical procedure. Therefore, it is crucial that the performance of the first incision be precise, balanced, and symmetrical, while applying an equal measure of force and pressure along the entire path of the incision.

Furthermore, precision is crucial when closing the incision at the end of the procedure, so that the stitching or stapling of the skin tissue is done so that the same areas that were separated by the incision will be rejoined as accurately as possible. There is therefore a need for a device and a method that will enable performance of a first incision in the skin that will be controlled and precise, at a desired length, at a desired and uniform depth, in a precise location on the patient's body, in a straight leveled line, and enabling precise closure of the edges of the incision at the end of the surgical procedure.

In the past, various devices and methods have been proposed for performance of incisions in the skin, intended for examination of the patient's bleeding time, prior to commencement of a surgical procedure and for taking blood samples.

A device for making a skin incision is described in U.S. Pat. No. 3,902,475 of Gebb et al. FIGS. 1a and 1b of the prior art are isometric views in different directions of an embodiment of a device for making a skin incision 200 of the invention of Gebb et al.

The device for making a skin incision 200 includes a base plate 213, having a base plate slot 213a, a support member 214, having an arched groove 214a, is connected vertically to the base plate 213. An arc-shaped member 215, having an arched slot 215a is mounted in the groove 214a. A knife 220 is supported by the arc-shaped member 215 and partially located inside the base plate slot 213a.

In operation the base plate 213 is in touch with the skin and the arc-shaped member 215 is moving along the arched slot 215a, causing the knife 220 to make an incision in the patient's skin.

The structure of the device for making a skin incision 200 forces the knife 220 to move in an arched movement, therefore the depth of the incision created is not uniform. Likewise, the speed of the incision depends upon the manner of pressure applied by the surgeon's finger upon the arc-shaped member 215, and the result can be non-uniform and insufficient speed, and along with involuntary movement of the device for making a skin incision 200, the resulting incision may be not along a straight line. Therefore it is not surprising that the device was not intended to be used and was never used for performing the first incision in open surgeries but rather for testing patients' bleeding time.

FIG. 1c of the prior art is a side view of a scalpel 300, held in a hand 400 of a surgeon during performance of an incision 500.

The standard method, in use at present, of performing a first incision in the skin, is completely manual, with a scalpel 300 held in the surgeon's dominant hand 400 at a suitable angle relative to the patient's body, and performing a skin incision 500 with a pulling motion. Seeing as the skin incision 500 is done with a free hand, it is difficult for the surgeon to perform it in a precise, symmetrical, balanced, leveled and consistent manner with regard to the path, position and the force applied, and it is difficult, nearly impossible, to achieve a uniformly desired depth and an accurate length.

In most cases the surgeon is positioned perpendicular to the patient's body, therefore, the angle from which the surgeon is viewing the patient's body also makes it difficult to form a straight and accurately positioned incision on the body. Other difficulties for the surgeon are that there is no support for the hand performing the incision and that the surface of the body is flexible and curved.

After completion of the surgical procedure, it is important to rejoin the edges of the incision precisely. At present, the standard practice is to close the incision by means of stapling the edges of the incision with a surgical stapler, or by stitching the edges of the incision while, when using a surgical stapler the edges of the incision are being held together by the surgeon's assistant with tweezers, and the surgeon uses the stapler to attach the edges of the incision to each other with staples. With these means and this method, it is difficult for the surgeon to be consistent and precise in arranging the skin as it was prior to the incision and also in locating the staples along the incision line. A common resulting effect of imprecise attachment of the edges of the incision is the creation of “dog ears” along and on the ends of the postoperative scar, and other inconsistencies in skin tensions, deviation in the scar line etc.

None of the prior art devices and methods of use are dealing with the challenges mentioned above and none of them comprise all of the characteristics and functions included in the present invention and its method of use.

There is therefore a need for a method and a device for producing a skin incision, which comprises a combination of all of the above characteristics and functions.

### BRIEF SUMMARY EMBODIMENTS OF THE INVENTION

The background art does not teach or suggest a method and a device for producing a skin incision, which enable the performance of a controlled and precise first incision, in a predetermined length, at a desired and uniform depth, at a precise location and orientation on the patient's body, in a straight line, with simultaneously created precise markings that facilitate a precise closure of the edges of the incision at the end of the surgical procedure, allowing for a supported positioning of the device used.

The present invention overcomes these deficiencies of the background art by providing a device, designed such that it is convenient to be held for producing a skin incision, the operation of which includes all or some of the following stages (not necessarily in the following order): selecting desired depth and length of incision there by cocking the device prior to performance of the procedure, removing a sealing protective cover, releasing a safety button, placing in the desired place on the patient's body, activating the device there by causing movement of the surgical blade with a swift, forced, and straight movement at a uniform depth, by force of stored energy that is released when the surgeon chooses to apply it while holding the device in place, to simultaneously create an incision that is at a predetermined length and depth and marking dots or lines in rows that are parallel to the incision line for precise closure.

As used herein above and below including in the specification and the claims sections, the term cutting and marking device (in short: “CM device”) refers to a device for producing a skin incision and markings along its sides according to the present invention.

Additional objectives and advantages of the invention will be set forth in part in the description which follows and, in part, will be obvious from the description, or may be learned by practice of the invention.

### BRIEF DESCRIPTION OF THE DRAWINGS

The invention is herein described, by way of example only, with reference to the accompanying drawings, wherein:

FIGS. 1a and 1b of the prior art are isometric views in different directions of an embodiment of a device for making a skin incision.

FIG. 1c of the prior art is a side view of a scalpel held in a surgeon's hand when performing an incision.

FIG. 2a is a top view schematic illustration of an illustrative, first embodiment of a CM device according to the present invention.

FIG. 2b is a top isometric schematic illustration of an illustrative, first embodiment of a CM device according to the present invention.

FIG. 2c is a front view schematic illustration of an illustrative, first embodiment of a CM device according to the present invention.

FIG. 2d is a right side view schematic illustration of an illustrative, first embodiment of a CM device according to the present invention.

FIG. 3 is an exploded isometric view schematic illustration of an illustrative, first embodiment of a CM device according to the present invention.

FIG. 4 is an isometric view schematic illustration of an illustrative, first embodiment of a body of a CM device, and of a sterile case according to the present invention.

FIG. 5 is an isometric view schematic illustration of a stopper of a first embodiment of a CM device according to the present invention.

FIG. 6a is an isometric view schematic illustration of a blade holder assembly of a first embodiment of a CM device according to the present invention.

FIG. 6b is an exploded isometric view schematic illustration of a blade holder assembly of a first embodiment of a CM device according to the present invention.

FIG. 6c is a right side view schematic illustration of the blade holder assembly of a first embodiment of a CM device according to the present invention.

FIG. 6d is a right side view schematic illustration of the blade holder assembly of a first embodiment of a CM device according to the present invention.

FIG. 6e is a top view schematic illustration of a blade holder assembly of a first embodiment of a CM device according to the present invention.

FIG. 7a is a right side view schematic illustration of a body left hand of a first embodiment of a CM device according to the present invention.

FIG. 7b is an isometric view schematic illustration of a first embodiment of a CM device according to the present invention, in a preliminary state, from which the body right half and the sealing cover have been removed.

FIG. 7c is a right side view schematic illustration of an illustrative, first embodiment, of a CM device according to the present invention, in a preliminary or post activation final state, from which the body right half has been removed.

FIG. 7d is a right side view schematic illustration of an illustrative, first embodiment, of CM device according to the present invention, in an engaged (cocked) state, from which the body right half and the sealing cover have been removed.

FIG. 7e is a right side view schematic illustration of a first embodiment of a CM device according to the present invention, in a state of movement of the blade holder assembly, from which the body right half and the sealing cover have been removed.

FIG. 7f is a right side view schematic illustration of a body right hand of a first embodiment of a CM device according to the present invention.

FIG. 8a is an isometric view schematic illustration of a marking and sealing assembly of a first embodiment of CM device according to the present invention, upon which the section plane 8b-8b is marked.

FIG. 8b is a schematic cross sectional view 8b-8b of the sealing cover cells, according to the present invention.

FIG. 8c is a rear view schematic illustration of a left markers base and a right markers base of the first embodiment of a CM device according to the present invention.

FIG. 9a is an isometric view schematic illustration of a triggering and locking assembly of a first embodiment of a CM device according to the present invention.

FIG. 9b is an exploded isometric view schematic illustration of a triggering and locking assembly of a first embodiment of a CM device according to the present invention.

FIG. 10 is an exploded isometric view schematic illustration of a second embodiment of a CM device according to the present invention.

FIG. 11a is a top view schematic illustration of the blade holder assembly of a second embodiment of a CM device according to the present invention, upon which the section plane 12a-12a is marked.

FIG. 11b is a side view schematic illustration of the blade holder assembly of a second embodiment of a CM device according to the present invention.

FIG. 11c is a front view schematic illustration of the blade holder assembly of a second embodiment of a CM device according to the present invention.

FIG. 11d is a side view schematic illustration of the blade holder assembly of the second embodiment of a CM device having an inclined blade holder cylinder according to the present invention.

FIG. 12a is a schematic cross sectional view 12a-12a of the blade holder assembly, of a second embodiment of a CM device according to the present invention.

FIG. 12b is an isometric view schematic illustration of the blade holder of a second embodiment of a CM device according to the present invention.

FIG. 12c is an exploded isometric view schematic illustration of the blade holder assembly of a second embodiment of a CM device according to the present invention.

FIG. 13a is a side view schematic illustration of the triggering and locking assembly of a second embodiment of a CM device according to the present invention.

FIG. 13b is a front view schematic illustration of the triggering and locking assembly of a second embodiment of a CM device according to the present invention.

FIG. 13c is an isometric view schematic illustration of the triggering and locking assembly of a second embodiment of a CM device according to the present invention.

FIG. 14a is a side view schematic illustration of the marking and sealing assembly of a second embodiment of a CM device according to the present invention.

FIG. 14b is an exploded isometric view schematic illustration of an illustration of the marking and sealing assembly of a second embodiment of a CM device according to the present invention.

FIG. 14c is a front view schematic illustration of the marking and sealing assembly of a second embodiment of a CM device according to the present invention.

FIG. 14d is an isometric view schematic illustration of the marker stamp unit of a second embodiment of a CM device according to the present invention.

FIG. 15a is a top view schematic illustration of a second embodiment of a CM device according to the present invention, upon which the section plane 15c-15c is marked.

FIG. 15b is an isometric view schematic illustration of the roller and segments of the energy accumulator of a second embodiment of a CM device according to the present invention.

FIG. 15c is a schematic cross sectional view 15c-15c of a second embodiment of a CM device according to the present invention.

FIG. 16a is a front view schematic illustration of the stopper of a second embodiment of a CM device according to the present invention.

FIG. 16b is a side view schematic illustration of the stopper of a second embodiment of a CM device according to the present invention.

FIG. 16c is a side view schematic illustration of a second embodiment of a CM device according to the present invention.

FIG. 16d is a back view schematic illustration of a second embodiment of a CM device according to the present invention.

FIG. 17a is an isometric view schematic illustration of a second embodiment of a CM device having a right side cover and a left side cover, according to the present invention.

FIG. 17b is an isometric view schematic illustration of a second embodiment of a CM device having a right side cover and a left side cover, according to the present invention, with the right side cover and the left side cover separate from the remaining parts of the CM device.

FIG. 17c is a front view schematic illustration of a second embodiment of a CM device having a right side cover and a left side cover, according to the present invention.

FIG. 17d is a top view schematic illustration of a second embodiment of a CM device having a right side cover and a left side cover, according to the present invention.

In order to leave no room for doubt, the elements shown in the illustrations of the present patent application are presented in a manner that enables understanding them clearly, and the scales, size relations, and shapes are not in any way limiting their embodiment.

It should also be noted that the definition of one embodiment as “first” and the other as “second” is not meant to indicate that the first is better than or superior to the second nor is it vice versa.

### DETAILED DESCRIPTION OF EMBODIMENTS OF THE INVENTION

To remove any doubt, note that the manner in which the elements of the present invention are described in the illustrations can be highly detailed, however it is not in any way limiting the present invention and it is for the purposes of clarification and furthering explication. The present invention can be implemented in embodiments that differ from the specification given with regard to the illustration.

The present invention is of a device and method for creating a first incision in skin during surgical procedures and for simultaneous marking along the edges of the incisions.

The principles and operation of the device and the method for creating a first incision in a patient's skin during surgical procedures and for simultaneous marking along the edges of the incision according to the present invention may be better understood with reference to the drawings and the accompanying description.

Before explaining embodiments of the invention in detail, it is to be understood that the invention is not limited in its application to the details of construction and the arrangement of the components set forth in the following descriptions or illustrations in the drawings.

Unless otherwise defined, all technical and scientific terms used herein have the same meaning as commonly understood by one of ordinary skill in the art to which this invention belongs. The materials, dimensions, methods, and examples provided herein are illustrative only and are not to be construed as limiting.

The following list is a legend of the numbering of the application illustrations: CM device
1ba back side
1fr front side
1ri right side
1le left side
1bw base width
1to top side
1bo bottom side
1t internal polygon (such as trapezoid)
1tf polygon front side
1tr polygon rear side
1tu polygon upper side
1tua polygon upper side wave shape
1tb polygon bottom side
10 body
10rh body right half
10lh body left hand
10lha body left hand bottom surface
10lhb body left hand track
10lhbl body left hand track length
10lhc body left hand tenon
10lhd body left hand stopper hole
10lhe body left hand pusher hole
10lhf body left hand roller pin
10lhg body energy accumulator pin
10lhh body left hand trigger hole
10lhi body left hand safety button hole
10lhj body left hand trigger side hole
10rh body right hand
10rha body right hand bottom surface
10rhb body right hand track
10rhc body right hand tenon
10rhd body right hand stopper hole
10rhe body right hand pusher hole
10rhf body right hand roller pin
10rhh body right hand trigger hole
10rhi body right hand safety button hole
10rhj body right hand trigger side hole
10u body upper segment
10lw lower part of the body width
10uw upper part of the body width
11a dot marker
11b marker stamp unit
11c marker stamp unit root
11d marker stamp unit base
11l left markers base
11la lateral distance
11lb dot marker length
11lo longitudinal distance
11r right markers base
12 pusher
12a pusher pivot
12b pusher arm
12c pusher base
12d pusher hole
13 roller
13a roller ring
14 stopper
14a stopper central part
14at stopper central part top
14b stopper side shelf
14bw stopper width
14c stopper vertical shelf
14d stopper pin
14e stopper bottom rail
14f stopper bumper
14g stopper main body
14h stopper handle
15 blade
15a blade sharp edge
15aa blade tip
15b blade upper end
15c blade niche
15d blade slot
15e blade pin
15f blade tip projection distance
16 blade holder
16a blade holder base
16aa blade holder base bottom surface
16b blade holder cylinder
16ba blade holder cylinder bottom surface
16c internal thread
16d blade holder slot
16e blade holder eye
16f blade holder first pin
16g blade holder second pin
16h blade holder elastic arm
16i elastic arm jag
16j blade holder protrusion
16k blade holder cogs
16m blade holder painted depth indication lines
16n blade holder projection
16p blade holder handle
16q blade holder bottom line
16r blade holder cylinder front line
17 depth regulator
17a depth regulator head
17b depth regulator groove
17c depth regulator bolt
17d depth regulator ring
17e depth regulator cogs
17f depth regulator body
17g depth regulator first arm
17h depth regulator second arm
17i depth regulator second arm projection
18 sealing cover
18a sealing cover base
18b sealing cover canal
18c sealing cover cell
18d marking and sealing assembly
19 trigger
19a trigger protrusion
19b trigger spring
19c trigger pivot
19d trigger main arm
19e trigger hole
19f trigger flexible joint
19g trigger handle
19h trigger side arm
20 safety button
21 energy accumulator
23 paint
24 sponge
30 sterile case
40 line marker
51r right side cover
51rb right side cover bottom external edge
51l left side cover
51lb left side cover bottom external edge
51n notch
150 blade holder assembly
190 triggering and locking assembly
200 prior art device for making skin incision
213 base plate (of the prior art device for making skin incision)
213a base plate slot (of the prior art device for making skin incision)
214 support member (of the prior art device for making skin incision)
214a groove (of the prior art device for making skin incision)
215 arc-shaped member (of the prior art device for making skin incision)
215a arched slot (of the prior art device for making skin incision)
220 knife (of the prior art device for making skin incision)
300 prior art scalpel
400 surgeon dominant hand
500 skin incision
501 planned incision depth
502 planned incision length
Alpha blade holder assembly angle when cocked to the back side
Beta angle between the blade holder bottom line and the blade holder cylinder front line

The present invention seeks to meet the requirement for performance of elongated incisions in open surgical procedures, when it is advantageous upon performing them freehanded.

Elongated incisions, as opposed to local incisions, which are actually punctures, for example, for the purpose of taking a blood sample or measuring the duration of hemorrhaging.

A geometrical indication for an elongated incision is the ratio between the width of the incision and its length. An incision can certainly be considered elongated if this ratio is larger than 100. The typical width of a surgical scalpel is approximately 0.4 mm, therefore, an incision of 4 or 5 cm clearly meets the definition of an elongated incision. Such incisions have a significant advantage with regard to the medical and aesthetic result when using the device and method of the present invention.

Hereinafter, embodiments of the present invention are explained in detail by referring to the drawings.

Note: different components are presented in the following illustrations in different scales in order to enable better views of significant details.

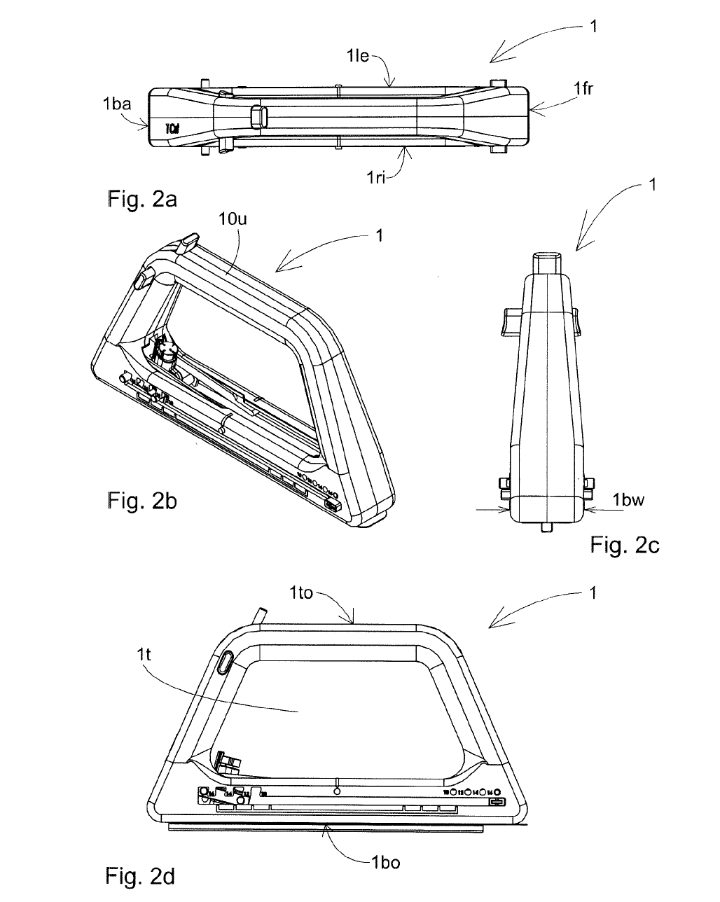

FIG. 2a is a top view schematic illustration of a first embodiment of a CM device 1 according to the present invention. The illustration defines sides of the CM device 1 in a manner that will facilitate further understanding of the invention; however these definitions are in no way limiting the present invention. The illustration shows the markings on the front side 1fr, the back side 1ba, the right side 1ri, and the left side 1le.

FIG. 2b is an isometric schematic illustration of a first embodiment of a CM device 1 according to the present invention. The upper part of the device 1 has a body upper segment 10u, which can serve as one of the segments that are easy to hold by the surgeon.

FIG. 2c is a front view schematic illustration of a first embodiment of a CM device 1 according to the present invention. The illustration is marked with base width 1bw.

FIG. 2d is a right side view schematic illustration of a first embodiment of a CM device 1 according to the present invention. The illustration defines surfaces of the CM device 1 for the purpose of facilitating further understanding of the invention; however these definitions are in no way limiting the present invention.

The illustration shows the markings of the top side 1to and the bottom side 1bo, and an internal polygon 1t. The internal polygon 1t serves as a place where the surgeon's fingers can be situated when holding the CM device 1.

According to the first embodiment the bottom side 1bo as shown in the present illustration, is a straight line representing a flat surface, however other forms are possible, such as a slightly concave line, from a side view, representing a concave surface, all for the purpose of optimal conformity to the organ undergoing surgery.

According to the first embodiment the polygon 1t has a figure of a trapezoid, however the polygon may have other shapes, such as an inverted trapezoid, a rectangle, a square, or even shapes with more or less than four sides, and likewise the sides needn't necessarily be straight.

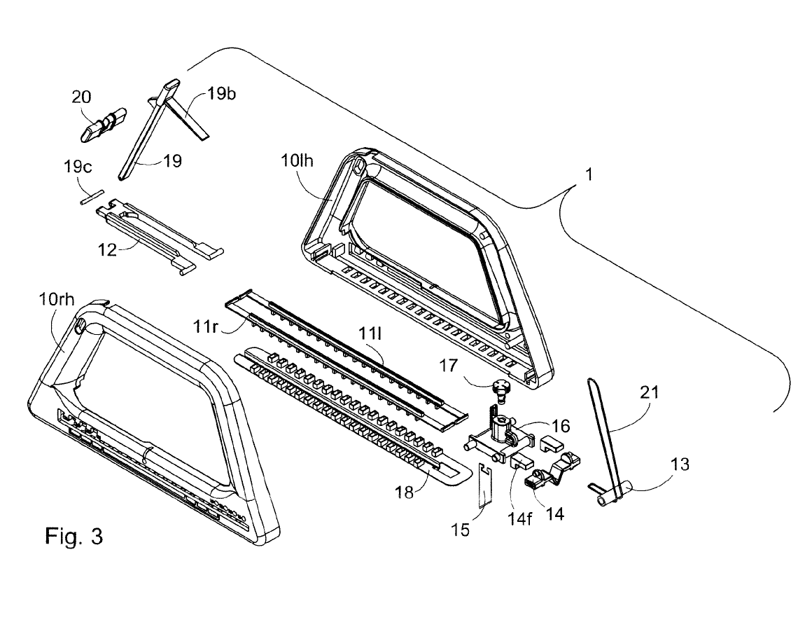

FIG. 3 is an exploded isometric view schematic illustration of the first embodiment of a CM device 1. The illustration shows the components of the CM device 1 first embodiment, which are the body right half 10rh component, the body left hand 10lh component, a left marker base 11l, a right marker base 11r, a pusher 12, a trigger spring 19b, a trigger pivot 19c, a roller 13, a stopper 14, a stopper bumper 14f, a blade 15, a blade holder 16, a depth regulator 17, a sealing cover 18, a trigger 19, a safety button 20, and an energy accumulator 21, which in the first embodiment is an elastic band, such as a rubber band however in other embodiments can be of other kinds, such as a spring or pump mechanism etc.

Note: according to the first embodiment, each of the components shown in the present illustration is composed as one piece, however this is in no way limiting the present invention, and any of the components may be composed of two or more interconnected parts.

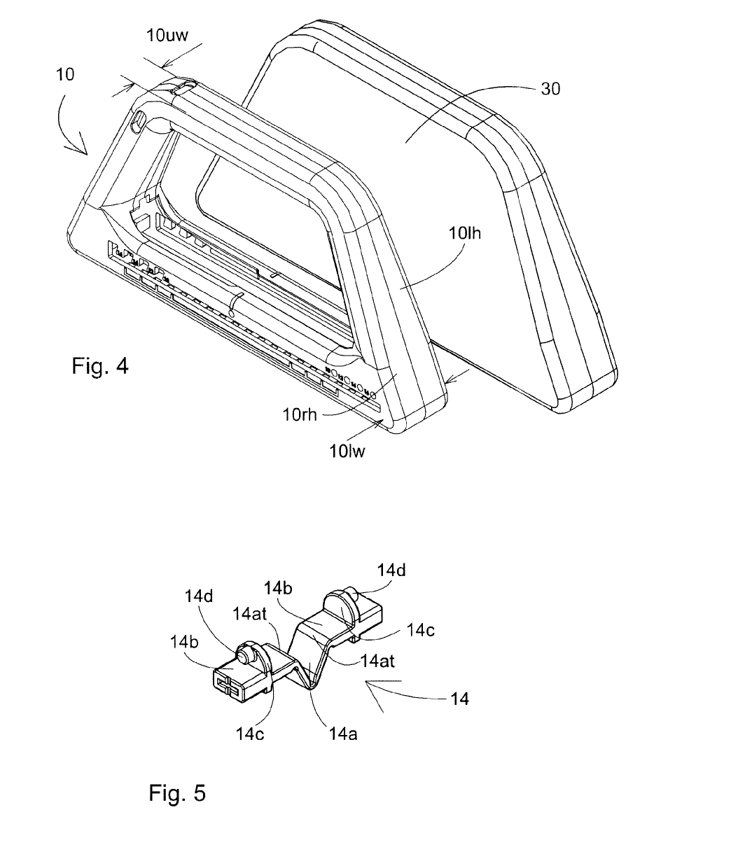

FIG. 4 is an isometric view schematic illustration of a body 10 of the first embodiment of the CM device 1, and of a sterile case 30, according to the present invention. The body 10 includes the body right half 10rh and the body left half 10lh, and serves as a casing containing the remaining components in their entirety, with the trigger 19 and the blade 15 protruding from it at various stages of action, while the sealing cover 18, which is attached to its bottom part, is removed from it prior to performance of the incision. (The last three components mentioned here are not shown in the present illustration, however were shown in FIG. 3, and will be shown in consequent illustrations.)

The body width can be maximal at the bottom part of the body 10, and can be a width that is particularly convenient for the surgeon to hold. A good example for width of the lower part of the body 10lw can be four centimeters, and a good example for the upper part of the body width 10uw can be two centimeters.

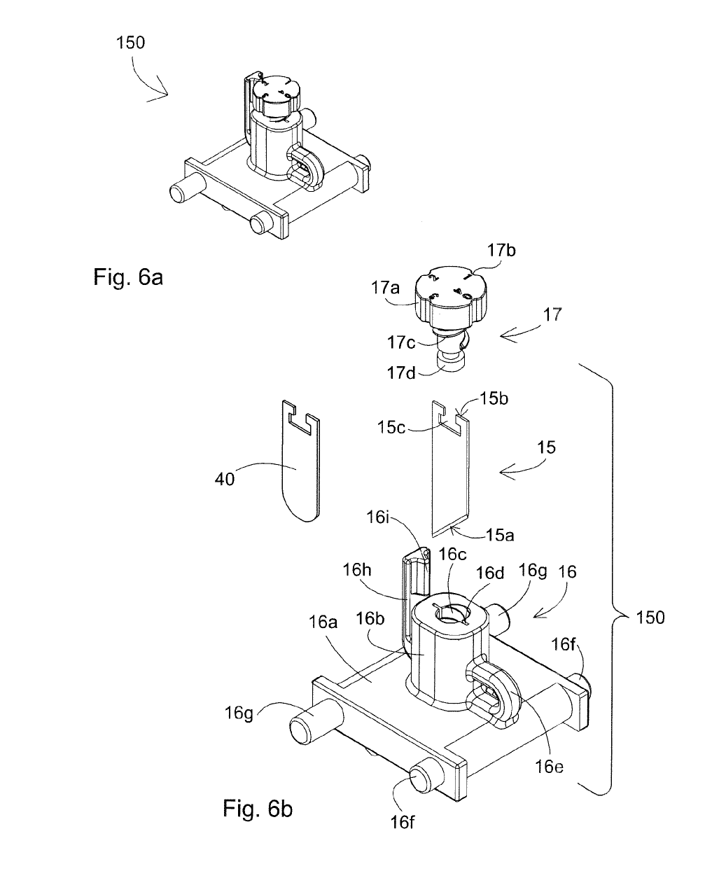

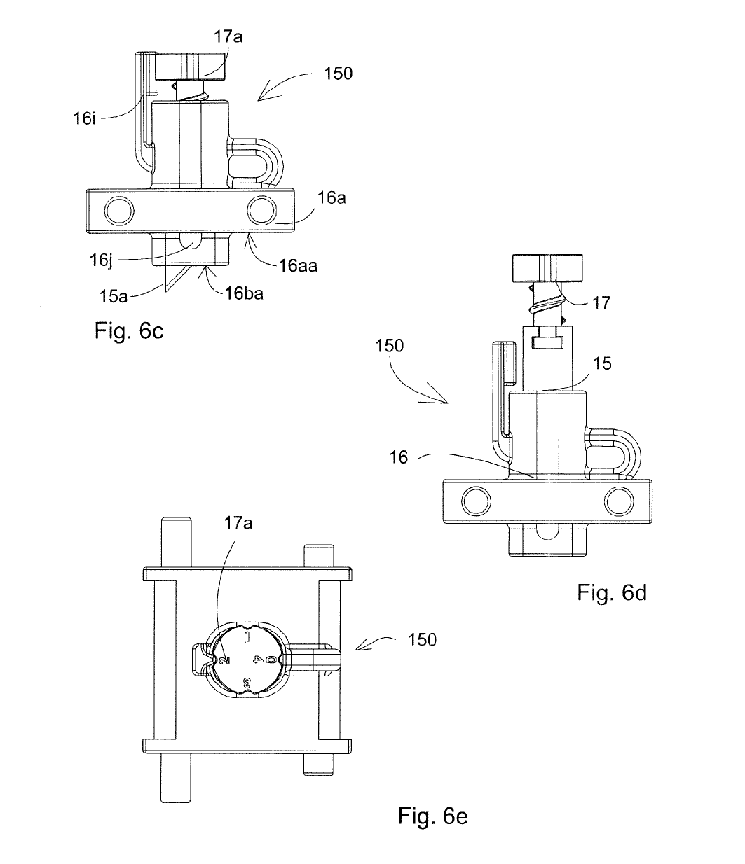

FIG. 5 is an isometric view schematic illustration of a stopper 14, of the first embodiment of the CM device 1 according to the present invention. As will be further explained, stopper 14 is designated to limit and stop the movement of the blade holder assembly 150 (not shown in the present illustration, shown in FIG. 6a), and thus determine where the incision ends.

The stopper 14 is made of elastic material and can include a stopper central part 14a, designed for example in a V-shape, granting it retractable force. Each stopper central part top 14at has a stopper side shelf 14b protruding from it. On each stopper side shelf 14b is a stopper vertical shelf 14c from which protrudes a stopper pin 14d.

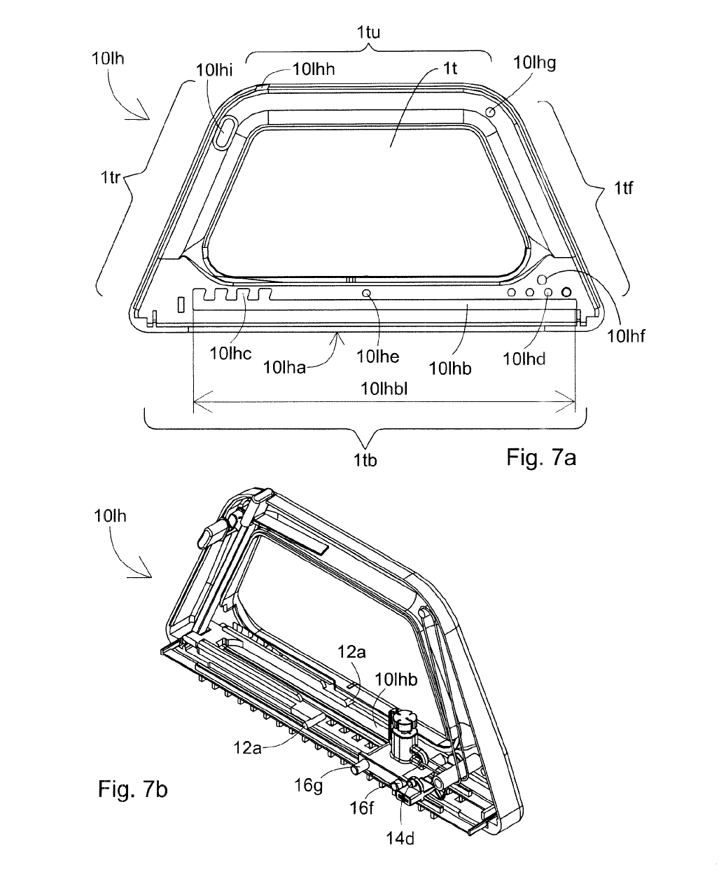

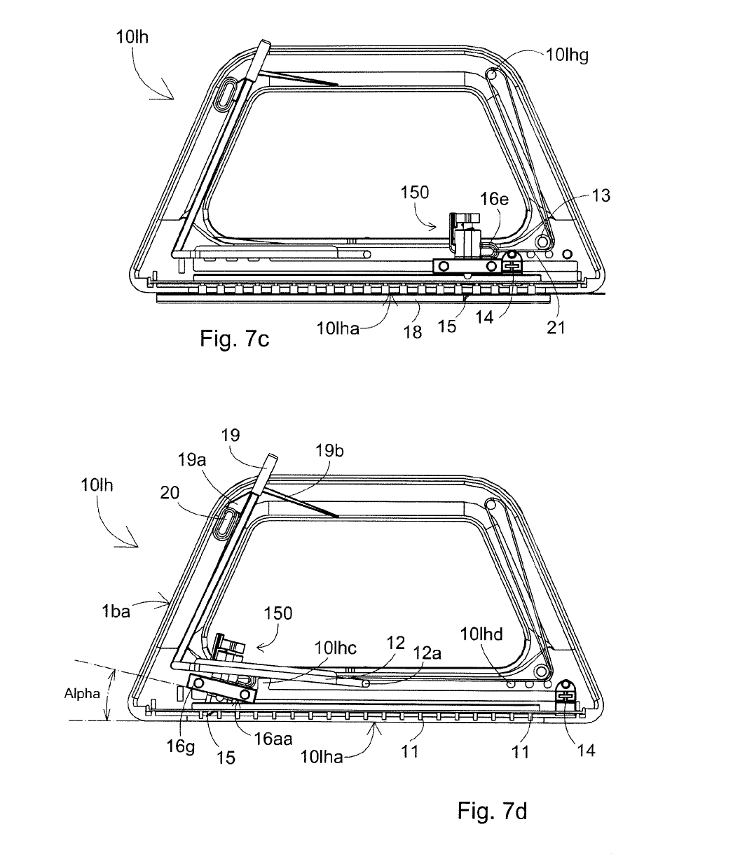

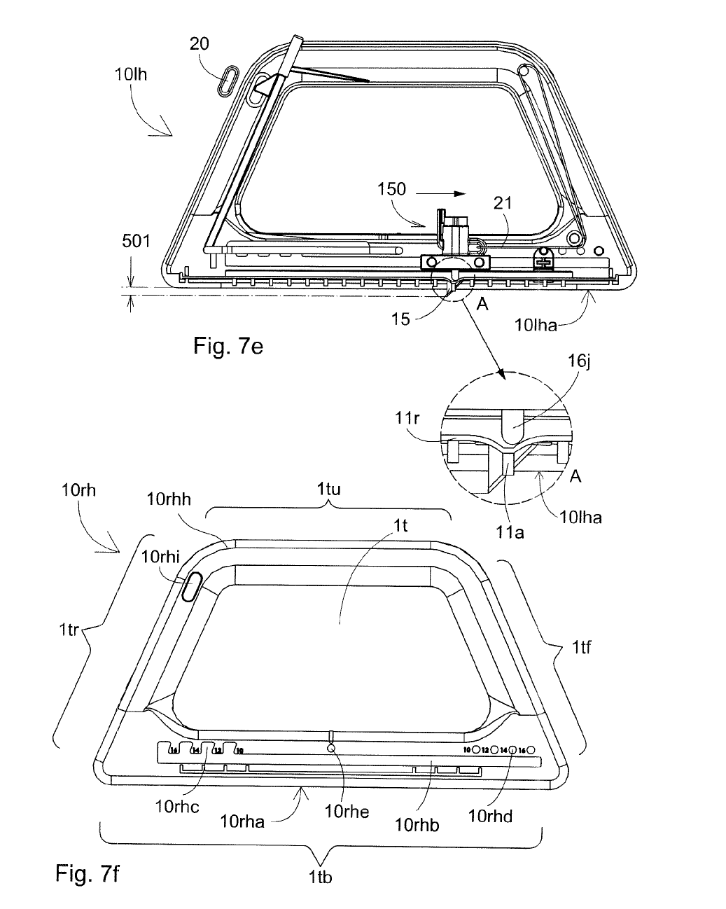

The two stopper pins 14d are usually each within a hole, such as the left hand stopper holes 10lhd (not shown in the present illustration, shown in FIG. 7a). In order to remove them from these holes and dispose them in other holes, it is necessary to press in from the sides towards the center on the two stopper side shelves 14b.

FIG. 6a is an isometric view schematic illustration of a blade holder assembly 150, of the first embodiment of a CM device 1 according to the present invention.

FIG. 6b is an exploded isometric view schematic illustration of a blade holder assembly 150, of the first embodiment of a CM device 1 according to the present 5 invention. The blade holder assembly 150 includes the blade 15, the blade holder 16, and the depth regulator 17; and it is designated to carry the blade 15 during performance of the incision to the patient's skin and to determine the depth of the incision.

The depth regulator 17 includes a depth regulator head 17a alongside several depth regulator grooves 17b, four according to the present embodiment, while each one can be marked with a number indicating the corresponding depth of the incision, for example 0, 1, 2, 3, and 4 (millimeter).

From the center of the depth regulator head 17a protrudes downward a depth regulator spiral bolt 17c, which is designated to move the blade 15 in linear movements downward or upward, to a certain distance which is suited to the desired depth of the incision, for each specific rotation angle of the depth regulator 17 and which is, for example according to the present embodiment, one millimeter for every quarter rotation.

Beneath the bottom end of the depth regulator bolt 17c is a depth regulator ring 17d designated to bear the blade 15. In close proximity to the blade upper end 15b is a blade niche 15c, the shape of which conforms to the shape of the depth regulator 17, particularly to the shape of the depth regulator ring 17d, so that the depth regulator ring 17d which is partially disposed within the blade niche 15c can rotate within it, and as it rises or drops relative to the blade holder 16, so does the blade 15 rise or drop accordingly.

The blade holder 16 includes a blade holder base 16a from whose upper part protrudes upwards a blade holder cylinder 16b containing an internal spiral thread 16c and a blade holder slot 16d. The shape and dimensions of the internal thread 16c conform to those of the depth regulator bolt 17c, while the dimensions of the blade holder slot 16d, which also passes through the blade holder cylinder 16b, conform to those of the blade 15.

Likewise, the blade holder 16 also includes a blade holder eye 16e, designated to serve as a grasping element for energy accumulator 21 (not shown in the present illustration, shown in FIG. 3), two blade holder first pins 16f, and two blade holder second pins 16g, all protruding from the sides of the blade holder base 16a, as shown in the illustration, and a blade holder elastic arm 16h at the end of which is an elastic arm jag 16i.

The blade holder elastic arm 16h extends, in the embodiment shown in the present illustration, from blade holder cylinder 16b and raises parallel to it. The elastic arm jag 16i conforms in shape and dimensions to serve as a stopper within a depth regulator groove 17b.

In blade holder assembly 150, the blade 15 can be replaced with a line marker 40, so that instead of performing an incision, a line will be marked for the purpose of later incision.

FIG. 6c is a right side view schematic illustration of a blade holder assembly 150 of the first embodiment of a CM device 1 according to the present invention. The blade holder base 16a has a blade holder base bottom surface 16aa, which according to the first embodiment of the CM device 1 is a planar surface.

Underneath the blade holder base bottom surface 16aa is a blade holder protrusion 16j. The elastic arm jag 16i is engaged within the depth regulator head 17a, and the blade sharp edge 15a protrudes downwards from the blade holder cylinder bottom surface 16ba.

FIG. 6d is a right side view schematic illustration of an illustrative, first embodiment of a blade holder assembly 150, of a CM device 1 according to the present invention. This illustration shows how the depth regulator 17 and the blade 15 are engaged with each other, when, for the purpose of demonstration, they have been lifted upward relative to the blade holder 16.

FIG. 6e is a top view schematic illustration of a blade holder assembly 150 of the first embodiment of a CM device 1 according to the present invention. This illustration shows numbers, expressing the desired incision depth, marked on the depth regulator head 17a.

FIG. 7a is a right side view schematic illustration of body left hand 10lh of the first embodiment of a CM device 1 according to the present invention. The body left hand 10lh is made of solid material and the body 10 as whole serves as a casing for the purpose of storing components, some of which have a capacity to move with regard to it, and some are fixed with regard to it. The body left hand 10lh shape, according to the present embodiment, is a polygon shape such as a trapezoid shape, encasing the internal polygon 1t. This polygon shape has a polygon front side 1tf, a polygon rear side 1tr, a polygon upper side 1tu, and a polygon bottom side 1tb.

Note that this trapezoid shape is in no way limiting the present invention and indeed will be of other dimensions in embodiments aimed at performing shorter in length incisions of, for example, 8 cm and less, in which the upper body segment that will remain in much the same size, will be longer than the lower body segment that will be shorter in length in comparison to the first embodiment illustrated in the present drawings.

According to the present invention, other embodiments of the CM device 1 can also be without ribs encasing internal polygon 1t, with a different solution meeting the need for convenient grip in the surgeon's hand of the CM device 1. This illustration shows numbers, expressing the desired incision depth, marked on the depth regulator head 17a.

The present illustration shows that the body left half 10lh also includes body left hand bottom surface 10lha, body left hand track 10lhb, body left hand tenons 10lhc, body left hand stopper holes 10lhd, body left hand pusher hole 10lhe, body left hand roller pin 10lhf, body energy accumulator pin 10lhg, body left hand trigger hole 10lhh, and body left hand safety button hole 10lhi.

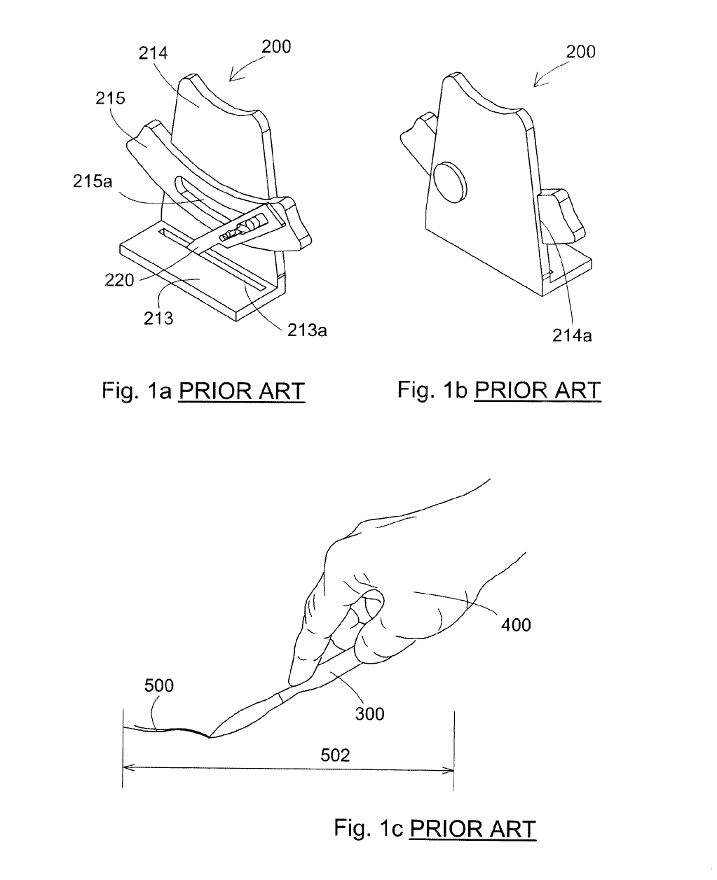

The body left hand track 10lhb has a body left hand track length 10lhbl. The left hand track length 10lhbl must be sufficiently long to perform the planned incision length 502, (not shown in the present drawing, shown in FIG. 1c).

Even though the present illustration shows four body left hand tenons 10lhc and four body left hand stopper holes 10lhd, this is in no way limiting the present invention, and there may be more or less of them in changing or in fixed distances between them.

Near each of the body left hand tenons 10lhc and the body left hand stopper holes 10lhd on the outer wall, a number can be marked, expressing the length of the incision that will be received from selecting the combination of both.

A similar structure, which appears as a mirror image, is on the body right half 10rh (not shown in the present illustration, shown in FIG. 7f).

FIG. 7b is an isometric view schematic illustration of the first embodiment, of a CM device 1 according to the present invention, in preliminary or final stage, from which the body right half 10rh, and the sealing cover 18 have been removed.

One of the two pusher pivots 12a is engaged in its regular position within the body left hand pusher hole 10lhe (not visible in the present illustration), and one of the two stopper pins 14d (not visible in the present illustration), is engaged in one of the body left hand stopper holes 10lhd (also not visible in the present illustration).

One blade holder first pin 16f and one blade holder second pin 16g (not visible in the present illustration) are engaged within the body left hand track 10lhb.

The trigger 19 is connected to the pusher 12 by a trigger pivot 19c (not shown in the present illustration, shown in FIG. 3), which enables them to rotate relative to one another.

FIG. 7c is a right side view schematic illustration of the first embodiment of a CM device 1 according to the present invention, in a preliminary or final state, from which the body right half 10rh has been removed.

The energy accumulator 21, according to the first embodiment is a rubber band, which is hooked on the body energy accumulator pin 10lhg, passes underneath roller 13, and is hooked to the blade holder eye 16e, and thus pulls and sets the location of the blade holder assembly 150 close to the stopper 14.

In this preliminary or final state, the blade 15 protrudes beneath the body left hand bottom surface 10lha according to the depth to which it is set and at most touches without penetrating the sealing cover 18.

FIG. 7d is a right side view schematic illustration of the first embodiment, of a CM device 1 according to the present invention, in an engaged cocked state, from which the body right half 10rh and the sealing cover 18 have been removed.

The blade holder assembly 150 has been manually cocked to the back side 1ba and a blade holder second pin 16g has been slid in to and engaged in a body left hand tenon 10lhc (not visible in the present illustration), which has been selected according to the desired length of the incision to be performed. This forms angle Alpha, ensuring that the blade 15 will not protrude beneath the body left hand 10lh, even when it is at its lowest relative to the blade holder assembly 150.

The trigger spring 19b pushes and holds the trigger 19 upward. In this state, a trigger protrusion 19a of the trigger 19, is positioned above a safety button 20 that prevents the trigger 19 from moving down even if an attempt to push it down is made.

The stopper 14 is engaged with selected body left hand stopper holes 10lhd and 10rhd (both not visible in the present illustration).

The body left hand and right tenons 10lhc and 10rhc and the body left and right hand stopper holes 10lhd and 10rhd in which the blade holder assembly 150 and the stopper 14 are engaged are selected according to the desired incision length.

One good option is to design and manufacture the body left hand 10lh so that there is conformity with the location of each one of the body left hand tenons 10lhc and the body left hand stopper holes 10lhd upon the body left hand 10lh and the distances between every two adjacent body left hand tenons 10lhc will correspond with the distances between every two adjacent body left hand stopper holes 10lhd so that the center of the incision will be at the center of the body left hand 10lh thus enabling the surgeon to intuitively locate the CM device 1 at a centered position on a patient's body surface when such a position is desired.

Note: the above given description will apply accordingly also to the body right half 10rh (not shown in the present illustration, shown in FIG. 4). However, it is clarified that the present invention is not limited to the above, which pertains only to the present embodiment, and the present invention includes, for example as few as one body left hand tenon 10lc and no stopper holes 10lhd or a few stopper holes 10lhd, and an incision line that is not central to the body left and right, which can be suitable for certain types of surgical procedures. At the bottom part of the body left hand 10lh are markers 11.

FIG. 7e is a right side view schematic illustration of the first embodiment, of a CM device 1 according to the present invention in a state of movement of the blade holder assembly 150, from which the body right half 10rh and the sealing cover 18 have been removed. The illustration marks detail A in a circle, which is magnified in the circle on the lower right side of the illustration.

After pushing the safety button 20 sideways and pushing down the trigger 19, the blade holder assembly 150 is released and pulled in the direction of the arrow marked above it by force of the energy accumulator 21.

The blade 15 moves with its end at a fixed distance (depth) beneath the body left hand bottom surface 10lha, which shall be referred to as a planned incision depth 501.

During performance of the incision, when the blade holder assembly 150 moves over the markers 11a, the blade holder protrusions 16j press respectively on the left markers base 11l and the right markers base 11r, which are made of flexible material, and they bend downward so as that pairs of markers 11a on the right and on the left, protrude slightly underneath the body left hand bottom surface 10lha and right hand bottom surface 10rha (not shown in the present illustration).

The shape of the blade holder protrusion 16j shown in the present illustration is not the only possible shape and is in no way limiting the present invention.

FIG. 7f is a right side view schematic illustration of a body right hand 10rh of the first embodiment of a CM device 1 according to the present invention. The present illustration shows that the body right half 10rh also includes body right hand bottom surface 10rha, body right hand track 10rhb, body right hand tenons 10rhc, body right hand stopper holes 10rhd, body right hand pusher hole 10rhe, body right hand trigger hole 10rhh, and body right hand safety button hole 10rhi.

Even though the present illustration shows four body right hand tenons 10rhc and four body right hand stopper holes 10rhd, this is in no way limiting the present invention, and there may be more or less of them in changing distances between them.

Near each of the body right hand tenons 10rhc and the body right hand stopper holes 10rhd on the outer wall, a number can be marked, expressing the length of the incision that will be received from selecting the combination of both.

This illustration also shows the sides of the trapezoid, as were shown in FIG. 7a.

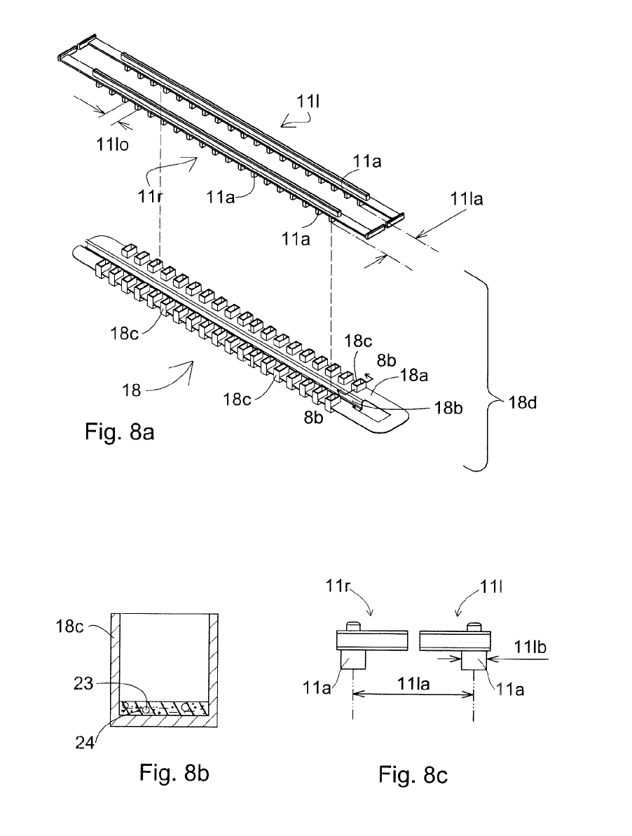

FIG. 8a is an isometric view schematic illustration of a marking and sealing assembly 18d of the first embodiment of a CM device 1 according to the present invention, upon which the section plane 8b-8b is marked.

The left markers base 11l and the right markers base 11r each include a straight line of dot markers 11a. At the end of each dot marker 11a is paint. Between the lines, which are parallel, there is a lateral distance 11la, namely at half of this distance, a row of colored dots will be marked on the patient's skin on both sides of the incision.

The shape of the dot markers 11a determines the shape of the markings made on the patient's skin, and they can be dots, dashes, V-chevrons, etc. Between every two adjacent dot markers 11a in one row, there is a longitudinal distance 11lo, (center to center of the markers 11a).

The left markers base 11l and the right markers base 11r can be made of a flexible material and when they are pressed towards the patient's body, during performance of the incision, when the blade assembly 150 (not shown in the present illustration, shown in part in FIG. 7d) moves over them, they bend periodically downward and the dot markers 11a mark the skin according to the dimensions of the lateral distance 11la and the longitudinal distance 11lo, which will later facilitate rejoining the edges of the incision after the surgical procedure is completed.

The sealing cover 18 includes a sealing cover base 18a, along the majority of which is a sealing cover canal 18b, which enables movement within of the blade 15 (not shown in the present illustration). Upon the sealing cover base 18a are two rows of sealing cover cells 18c whose location conforms to the location of the markers 11a. In a state prior to activation, each sealing cover cell 18c contains a dot marker 11a.

In the first embodiment shown in the present illustration, the sealing cover cells 18c protrude partially upward from the sealing cover base 18a.

FIG. 8b is a schematic cross sectional view 8b-8b of the sealing cover cells 18c, according to the present invention. The sealing cover cells 18c contain sponge 24, or another suitable material, containing paint 23, which serves the purpose of marking dots on the patient's skin.

FIG. 8c is a rear view schematic illustration of a left markers base 11l and a right markers base 11r of the first embodiment of a CM device 1 according to the present invention. An optimal typical dimension of the lateral distance 11la is 12.5 millimeters. This distance is measured between the centers of both markers 11a as shown in the present illustration, and it is equal to the dimension between the inner edge of one of them to the outer edge of the other one as shown in FIG. 8c. An optimal typical dimension of a dot marker length 11lb is 3.5 millimeters. However, other dimension values are possible and the present invention is in no way limited to these dimensions.

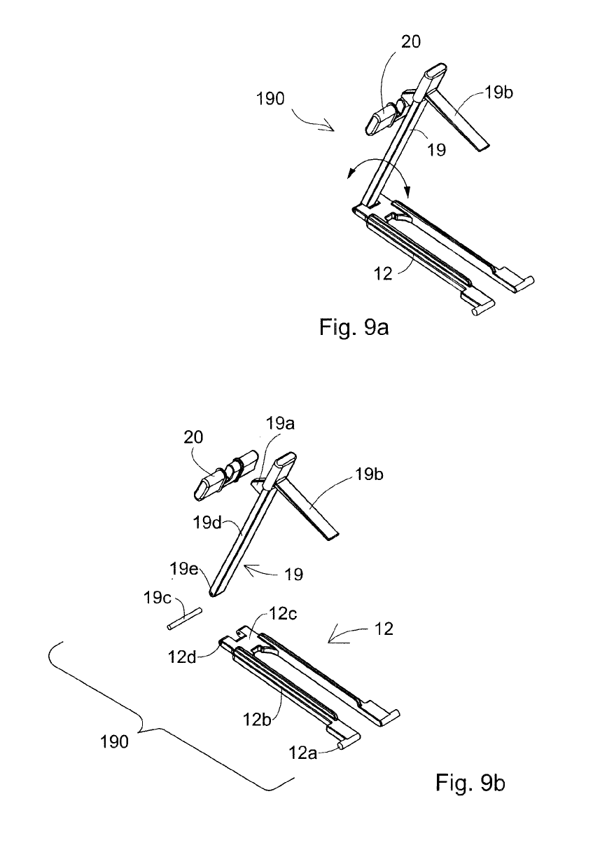

FIG. 9a is an isometric view schematic illustration of a triggering and locking assembly 190 of the first embodiment of a CM device 1 according to the present invention. The triggering and locking assembly 190 includes the pusher 12, the trigger 19, the trigger spring 19b and the trigger pivot 19c (not shown in the present illustration, shown in FIG. 9b). Between the pusher 12 and the trigger 19 there is a certain degree of freedom for rotational movement, as the double-headed arrow shows in the illustration.

FIG. 9b is an exploded isometric view schematic illustration of the triggering and locking assembly 190 of the first embodiment of a CM device 1 according to the present invention.

The pusher 12 includes a pusher base 12c, which according to the first embodiment has a U shape, when each of the U-arms has a pusher hole 12d. The two pusher arms 12b at the end of each of which is a pusher pivot 12a, are connected to the pusher base 12c.

The trigger 19 includes a trigger main arm 19d, at the lower end of which is a trigger hole 19e. The trigger spring 19b is connected to the trigger main arm 19d, by rigid or non-rigid connection.

The safety button 20 has a shape which also conforms to the shape of the trigger protrusion 19a. The trigger pivot 19c connects the trigger 19 to the pusher 12 and grants them the necessary freedom of slightly arced up and down movement.

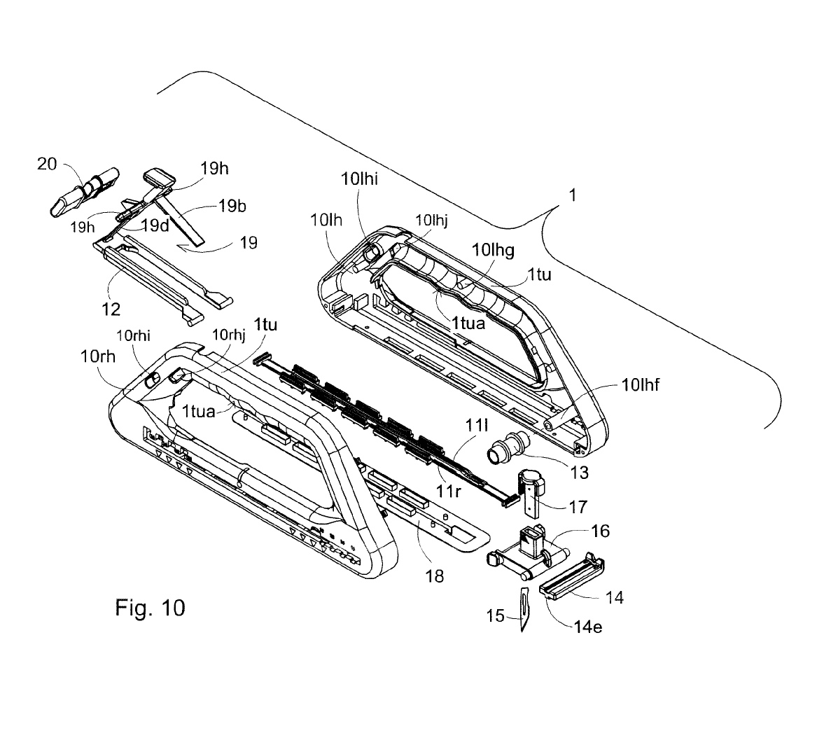

FIG. 10 is an exploded isometric view schematic illustration of a second 5 embodiment of a CM1 according to the present invention. The second embodiment of a CM device 1 differs in the structures of some components from those of its first embodiment. According to the present invention there may be various combinations of components from the first and the second embodiments of the CM device 1.

The illustration shows the components of a CM device 1 second embodiment, which are the body right half 10rh component, the body left hand 10lh component, a left marker base 11l, a right marker base 11r, a pusher 12, a trigger 19, a trigger spring 19b, while the last three components, as will be shown subsequently, are composed as a single integrated part, a roller 13, a stopper 14, a blade 15, a blade holder 16, a depth regulator 17, a sealing cover 18 and a safety button 20. The description given for the energy accumulator 21, of the first embodiment, (not shown in the present illustration), also applies to the second embodiment.

The illustration also shows that from the trigger main arm 19d, two trigger side arms 19h protrude. These enable activation of the trigger 19 by being pushed, as another option for activation.

After full assembly of the CM device 1 second embodiment, one trigger side arm 19h protrudes through a body left hand trigger side hole 10lhj, opened in the body left hand half 10lh, while the other trigger side arm 19h protrudes through a body right hand trigger side hole 10rhj, opened in the body right hand half 10rh. Also note that the shape of the stopper 14 of the second embodiment differs from that of the stopper 14 of the first embodiment and includes a stopper bottom rail 14e and also that the body left hand roller pin 10lhf is shaped as a hollow tube and is longer in the second embodiment than the body left hand roller pin 10lhf of the first embodiment.

Furthermore, the body energy accumulator pin 10lhg is disposed, according to the second embodiment, in proximity to the center of the polygon upper side 1tu, enabling use of a longer rubber band type energy accumulator 21 (not shown in the present drawing) than that of the first embodiment. This length is better for achieving an optimal ratio between the length of the rubber band type energy accumulator 21 when it is stretched i.e. when the CM device 1 is cocked and ready for activation, and its length before it is cocked or drawn or upon completion of the incision; a ratio determining the force that it generates at each stage of performing the incision.

Preferably, the rubber band type energy accumulator 21 should stretch to approximately twice its length. In order to enable convenient and easy manual grip, the polygon upper side 1tu is slightly wave shape in its center lower part 1tua.

Note: according to the second embodiment, each of the components shown in the present illustration is composed as one piece, however this is in no way limiting the present invention, and any of the components may be composed of two or more interconnected parts.

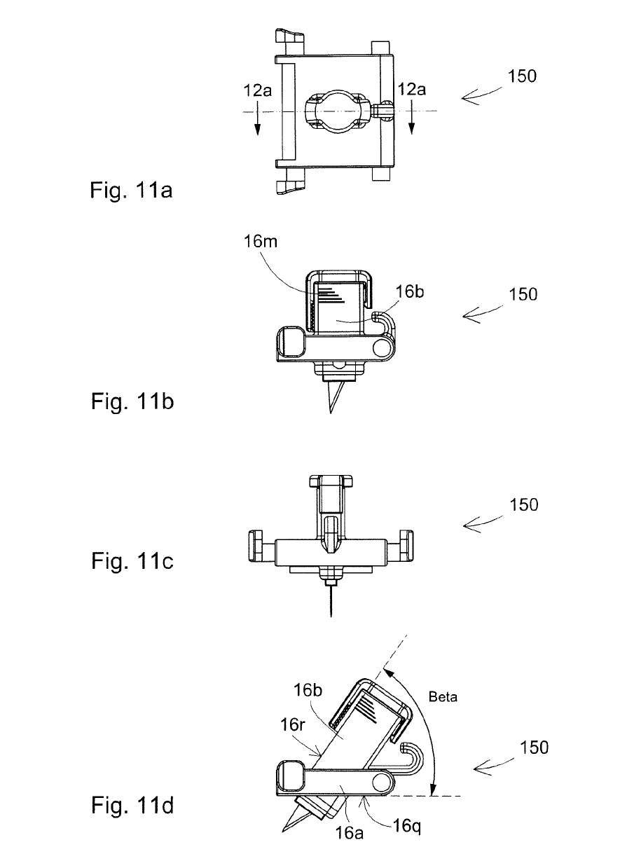

FIG. 11a is a top view schematic illustration of the blade holder assembly 150 of the second embodiment of a CM device 1 according to the present invention, upon which the section plane 12a-12a is marked.

FIG. 11b is a side view schematic illustration of the blade holder assembly 150 of the second embodiment of a CM device 1 according to the present invention. Upon the blade holder cylinder 16b there is a blade holder painted depth indication lines 16m designated to indicate the set depth by reference to a corresponding triangular mark on the blade holder first arm 17g (not marked in the present illustration).

FIG. 11c is a front view schematic illustration of the blade holder assembly 150 of the second embodiment of a CM device 1 according to the present invention. This view shows the blade holder assembly 150 in a symmetrical configuration, however other configurations are possible.

FIG. 11d is a side view schematic illustration of the blade holder assembly 150 of the second embodiment of a CM device 1 having an inclined blade holder cylinder 16b according to the present invention.

According to a variant of the second embodiment of a CM device 1 the blade holder cylinder 16b is slanted relative to the blade holder base 16a. For relative purposes, this slant is marked in the present illustration as angle Beta between the blade holder bottom line 16q and the blade holder cylinder front line 16r and is practically smaller than 90 degrees. This configuration is particularly effective when the beginning of the incision is close to a hard organ, such as the area of the knee downward, the elbow onward, etc., in a manner demanding that the incision start as close to the edge of the device as possible. In a variant of the second embodiment angle Beta is effectively 90 degrees.

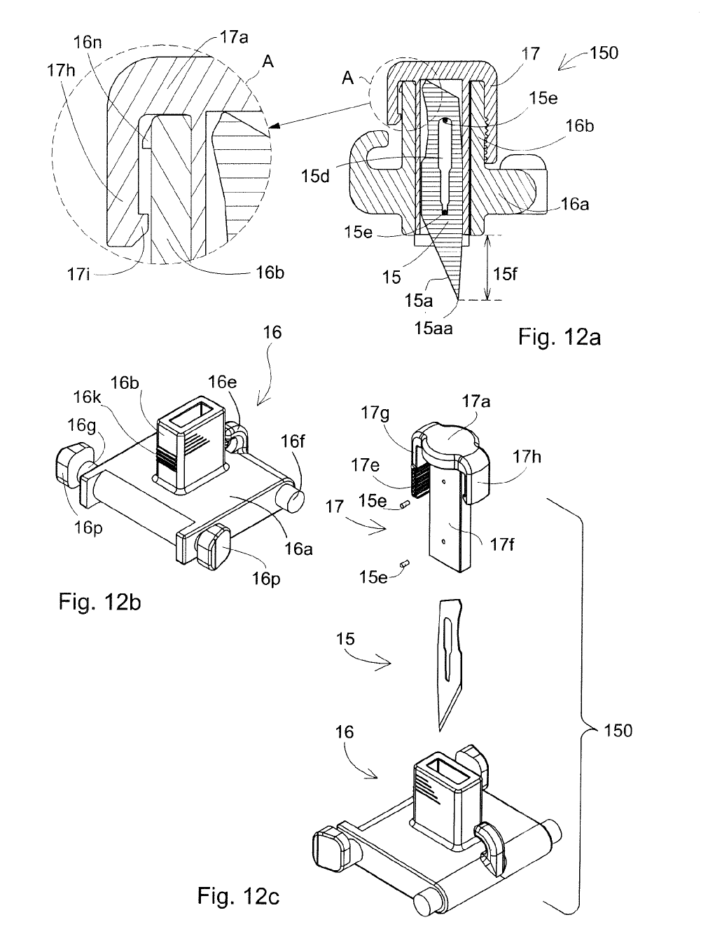

FIG. 12a is a schematic cross sectional view 12a-12a of the blade holder assembly 150, according to the present invention. The illustration marks detail A in a circle, which is magnified in the circle on the left side of the illustration.

The blade 15 is connected to the depth regulator 17 by means of one or more blade pins 15e, passing through a blade slot 15d. One other option for connection is adhesion of the blade 15 to the depth regulator 17, while yet another option is casting the depth regulator 17 on top of the blade 15 so that the blade 15 is casted within it These options enable connection of any type of blade 15 in use by surgeons and do not require a particularly designed blade as shown in the first embodiment.

Determining the extent of the downward protrusion of the blade 15 is done by placing the height of the depth regulator 17 relative to the blade holder cylinder 16b.

The blade holder cylinder 16b is a cylinder having a section shape such as a rectangular shape, from a top view, and the movement of the depth regulator 17 is done upward or downward relative to it as linear movement, without rotation. The movement is transmitted to the depth regulator 17 manually, by pushing down and pulling up. At the end of the depth regulator second arm 17h which is distant from the depth regulator head 17a from a depth regulator second arm projection 17i which is designated to prevent upward separation of the depth regulator 17 from the blade holder 16, with the assistance of a blade holder projection 16n.

The blade 15 has a blade sharp edge 15a and a blade tip 15aa wherein the blade tip projection distance 15f, from the blade holder base 16a, can be changed and controlled be the depth regulator 17.

FIG. 12b is an isometric view schematic illustration of the blade holder 16 of the second embodiment of a CM device 1 according to the present invention. From the blade holder base 16a protrudes upwards a blade holder cylinder 16b. Likewise, the blade holder 16 also includes a blade holder eye 16e, designated to serve as a grasping point for energy accumulator 21 (not shown in the present illustration, shown in FIG. 3), and two blade holder first pins 16f.

Upon the blade holder cylinder 16b are blade holder cogs 16k serving to prevent undesired movement between the depth regulator 17 (not shown in the present drawing), and the blade holder cylinder 16b. At the end of each of both of the blade holder second pins 16g there is a blade holder handle 16p and they both have spatial shapes which facilitate comfortable holding when pulling to trigger.

FIG. 12c is an exploded isometric view schematic illustration of the blade holder assembly 150 of the second embodiment of a CM device 1 according to the present invention. The depth regulator 17, the blade 15, and the blade holder 16 have been vertically separated.

The depth regulator head 17a is connected to the depth regulator body 17f, a depth regulator first arm 17g and a depth regulator second arm 17h. Upon the depth regulator first arm 17g there are depth regulator cogs 17e serving along with the blade holder cogs 16k (not shown in the present drawing), to prevent undesired vertical movement of the depth regulator 17 relative to blade holder 16.

The depth regulator head 17a has a shape, circular in the present illustration, however other shapes are also possible, enabling its being pulled upward and being pushed downward.

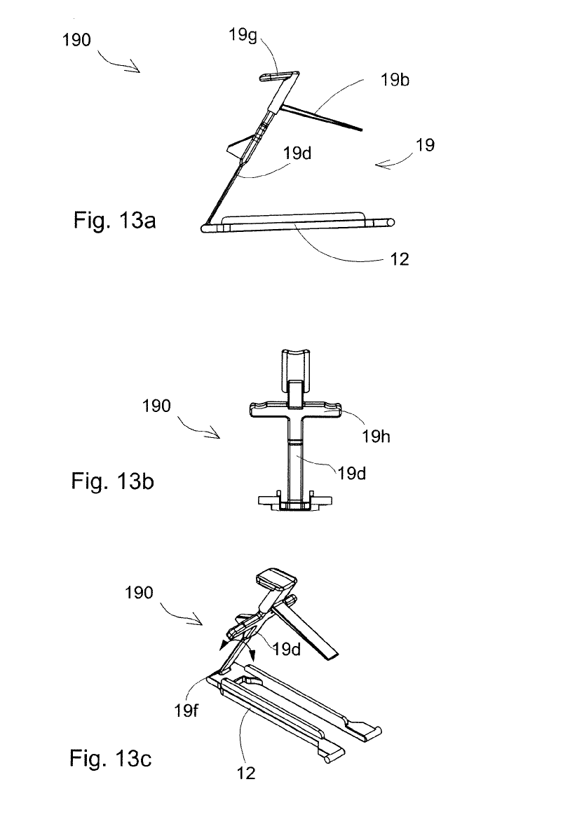

FIG. 13a is a side view schematic illustration of the triggering and locking assembly 190 of the second embodiment of a CM device 1 according to the present invention.

All components of the triggering and locking assembly 190, in the configuration shown in the present illustration, are composed as a single part, however the present invention is in no way limited to this, and the triggering and locking assembly 190 can be composed of separate parts.

The trigger 19 is connected, as noted, as a single part, to the pusher 12, the trigger spring 19b, and a trigger handle 19g. The trigger handle 19g is designated to facilitate the activation of the triggering and locking assembly 190 with the press of a finger.

FIG. 13b is a front view schematic illustration of the triggering and locking assembly 190 of the second embodiment of a CM device 1 according to the present invention. The trigger 19 is also connected to two trigger side arms 19h, also in order to facilitate the manual activation of the triggering and locking assembly 190.

FIG. 13c is an isometric view schematic illustration of the triggering and locking assembly 190 of a CM device 1 according to the present invention.

The trigger 19 is connected to the pusher 12 by means of a trigger flexible joint 19f, which is an area in which the dimensions, the shape, and the composing material provide sufficient flexibility to enable relative angular movement cycles between the trigger 19 and the pusher 12 without sustaining damage, in the direction shown by the marked arrows in both ends of the circular segment of the present illustration.

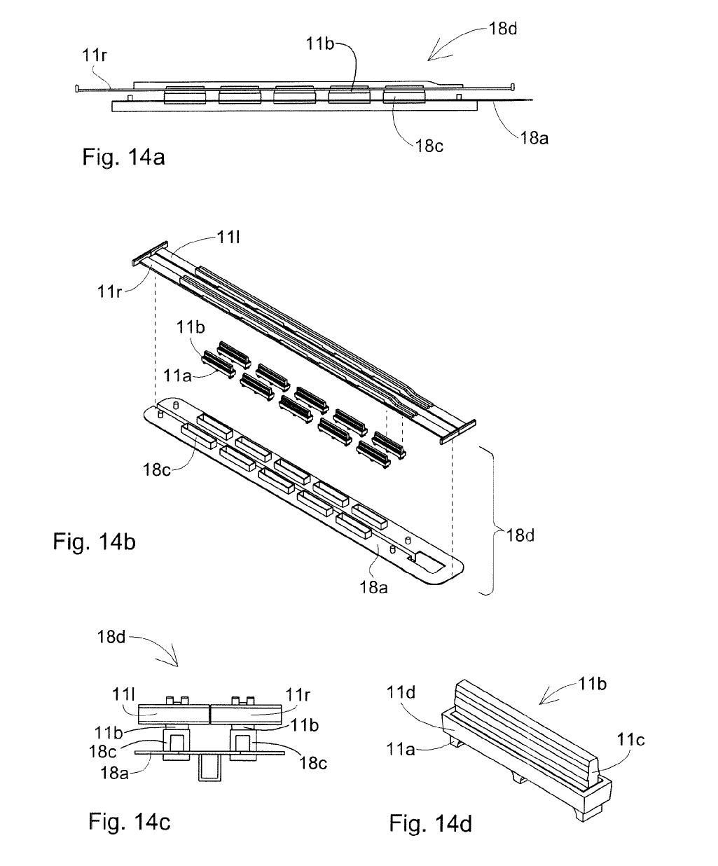

FIG. 14a is a side view schematic illustration of the marking and sealing assembly 18d of a second embodiment of a CM device 1 according to the present invention.

The right markers base 11r is connected to marker stamp units 11b, five in the configuration shown in the present illustration, however it is possible in a configuration with another number of stamp units and different sizes of marker stamp units. Each marker stamp unit 11b is encased within a sealing cover cell 18c connected to the sealing cover base 18a. The structure of the marking and sealing assembly 18d on the other side, not shown in the present illustration, is the same.

FIG. 14b is an exploded isometric view schematic illustration of an illustration of the marking and sealing assembly 18d of the second embodiment of a CM device 1 according to the present invention.

The marker stamp units 11b are shown in the present illustration as separated from the right markers base 11r and the left markers base 11l. From the bottom of each marker stamp unit 11b protrude dot markers 11a, three in the configuration shown in the present configuration; however configurations with other quantities are possible. Facing each marker stamp unit 11b is a sealing cover cell 18c connected to the sealing cover base 18a.

FIG. 14c is a front view schematic illustration of the marking and sealing assembly 18d of the second embodiment of a CM device 1 according to the present invention.

Each marker stamp unit 11b is connected to the right markers base 11r or to the left markers base 11l and is encased within a sealing cover cell 18c connected to the sealing cover base 18a.

The marker stamp units 11b collect paint 23 (not shown in the present drawing), stored within the sealing cover cells 18c similarly to the method described with regard to the first embodiment.

FIG. 14d is an isometric view schematic illustration of the marker stamp unit 11b of the second embodiment of a CM device 1 according to the present invention.

The marker stamp unit 11b has a marker stamp unit base 11d having an inverted truncated pyramid shape. This shape is designated to ensure a good seal upon contact with a sealing cover cell 18c (not shown in the present drawing), to prevent the evaporation of paint 23 (not shown in the present drawing).

The dot markers 11a protrude from the truncated end, which is shown turning downward in the present illustration. These can be at various quantities at various intervals, and even irregular intervals. The upward facing side of the marker stamp unit base 11d, according to the present illustration, has a protruding marker stamp unit root 11c enabling the connection of the marker stamp unit 11b to the to one of the marker bases (11r or 11l).

When cocking the CM device 1 (not shown in the present drawing) during movement of the blade holder assembly 150 (not shown in the present drawing), all marker stamp units 11b are pressed in turn toward the internal part of the sealing cover cell 18c wherein are pads 24 (not shown in the present drawing) soaked in paint 23 (not shown in the present drawing) within which it is already previously partially disposed and therefore it collects paint 23 (not shown in the present drawing),

After removal of the sealing cover 18, during performance of an incision, in the movement of the blade holder assembly 150 (not shown in the present drawing), each one of the marker stamp units 11b is pressed toward the patient's skin and makes the marking on it.

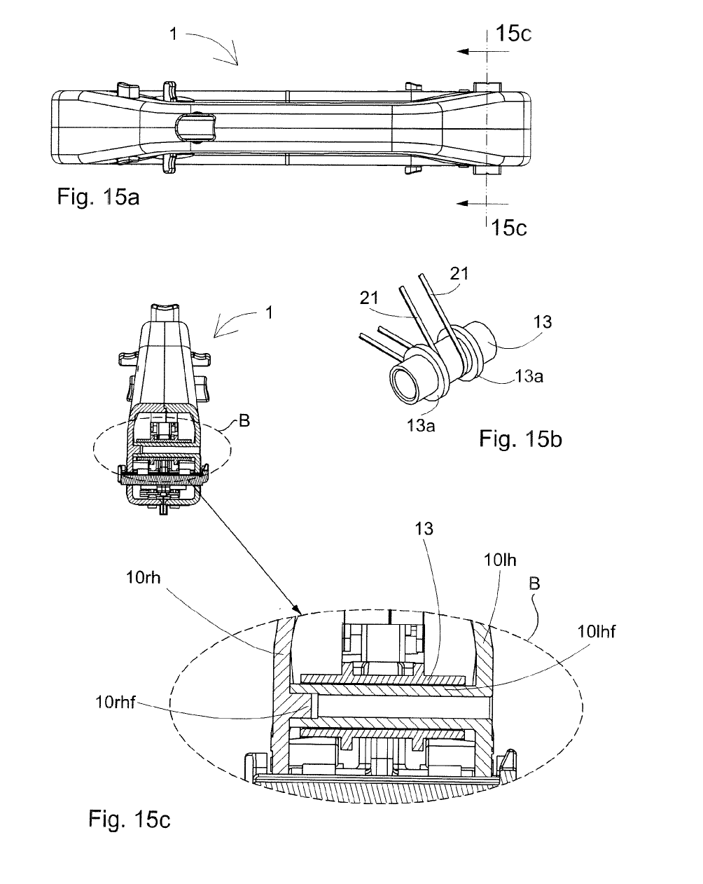

FIG. 15a is a top view schematic illustration of the second embodiment of a CM device 1 according to the present invention, upon which the section plane 15c-15c is marked.

FIG. 15b is an isometric view schematic illustration of the roller 13 and segments of the energy accumulator 21 of the second embodiment of a CM device 1 according to the present invention. The roller 13 is a hollow tube which has two roller rings 13a on its external side, preventing sideways sliding of the rubber band 21.

FIG. 15c is a schematic cross sectional view 15c-15c of the second embodiment of a CM device 1 according to the present invention.

The illustration marks detail B in an ellipse, which is magnified in the ellipse on the lower right side of the illustration.

The roller 13 is mounted upon the body left hand roller pin 10lhf which is connected to the body left hand 10lh, or both elements are composed as a single unit, which is at a length reaching the body right hand 10rh. The body right hand 10rh is connected or composed as a single part with a body left hand roller pin 10lhf which is short relative to the length of the body right hand roller pin 10rhf, and is contained within it, at its end. This structure provides a convenient option for assembly of the roller 13 and its subsequent activation.

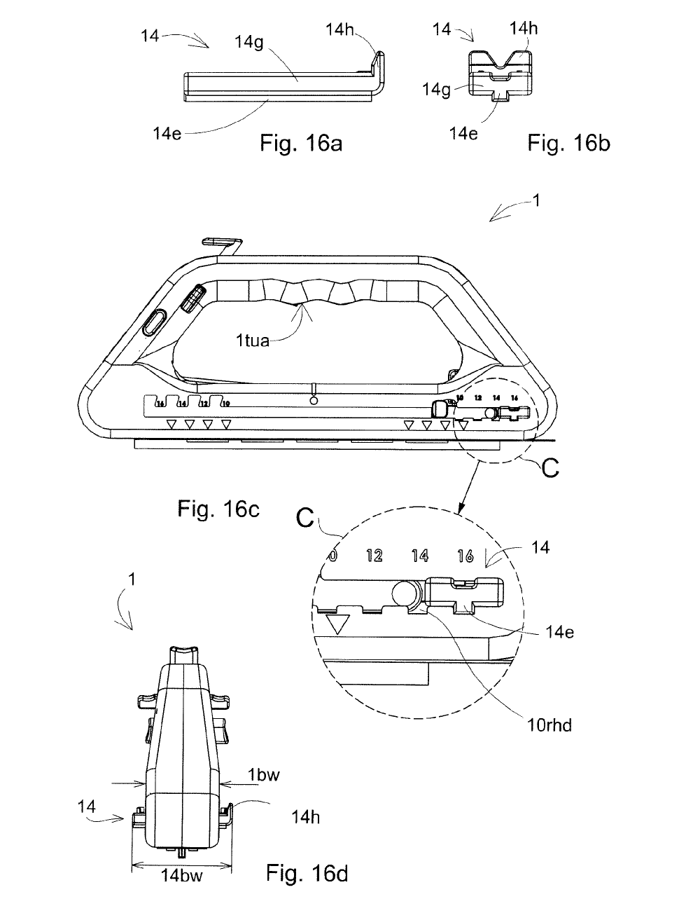

FIG. 16a is a front view schematic illustration of the stopper 14 of the second embodiment of a CM device 1 according to the present invention. The stopper 14 of the second embodiment has a stopper main body 14g at one end of which is a stopper handle 14h which facilitates its positioning, inserting it into the CM device 1 (not shown in the present drawing), and removing it. A stopper bottom rail 14e protrudes from the bottom of the stopper main body 14g.

FIG. 16b is a side view schematic illustration of the stopper 14 of a second embodiment of a CM device 1 according to the present invention. The stopper 14 includes a stopper main body 14g, a stopper handle 14h, and a stopper bottom rail 14e which in the present illustration are composed as a single unit, however according to the present illustration can also be made as separate components that are interconnected.

FIG. 16c is a side view schematic illustration of a second embodiment of a CM device 1 according to the present invention. The illustration marks detail C in a circle, which is magnified in the circle on the lower side of the illustration. The present illustration clearly shows the polygon upper side wave shape 1tua.

The stopper bottom rail 14e has a section, the shape of which conforms to the shape of the body right hand stopper hole 10rhd which is shown in the present illustration as a rectangle that is open upward. This enables positioning the stopper 14 in one of the body right hand stopper holes 10rhd. The same shape applies to body left hand stopper holes 10lhd, (not shown in the present drawing).

FIG. 16d is a back view schematic illustration of a second embodiment of a CM device 1 according to the present invention. The stopper handle 14h protrudes from one side of the device for producing a skin incision 1 while the other side of the stopper 14 protrudes from the other side. Namely, the stopper width 14bw is larger than the base width 1bw. The direction of the stopper 14 can be reversed and inserted from either right or left of the CM device 1, as preferred by the user.

Note: the CM device 1 can be designed and manufactured for single-time use or for repeated use.

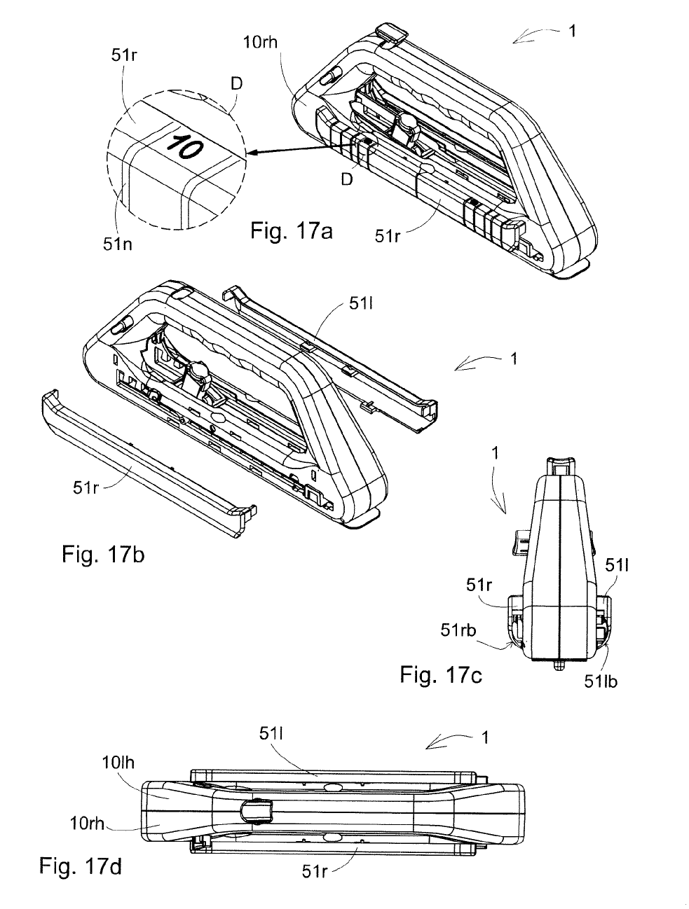

FIG. 17a is an isometric view schematic illustration of a second embodiment of a CM device 1 having a right side cover 51r and a left side cover 51l, according to the present invention.

The right side cover 51r and the left side cover 51l serve as bumpers which assist in preventing accidental contact with any component of the CM device 1, thus preventing any accidental activation when it is engaged, preventing contact with or pinching of the patient's skin by the blade holder 16 (not marked in the present drawing) during activation, and preventing slowing down, braking, or ceasing of the action of the blade holder 16, which may occur as a result of contact and friction with the patient's body, or any other disruptive element.

Furthermore, the right side cover 51r and the left side cover 51l may include indications that facilitate finding an accurate positioning on the patient body and boundaries of the incision to be made. The side covers may serve also as a ruler for measuring the length of the required incision. Furthermore, they grant the CM device 1 additional resistance to vertical pressure applied when the surgeon applies it to the patient's body, thus facilitating preservation of the body left hand track 10lhb and the body right hand track 10rhb (both not marked on the present drawing) in an open and undisturbed state, as is necessary for free movement of the blade holder 16.

The right side cover 51r and the left side cover 51l can also be made of a transparent material. The right side cover 51r and the left side cover 51l can be marked with notches 51n, or any other kind of marking, that can contribute to the performance of the actions that the CM device 1 is designed to perform.

The illustration marks detail D in a circle, which is magnified in the circle on the left side of the illustration. Notches 51n are marked on the right side cover 51r.

FIG. 17b is an isometric view schematic illustration of a second embodiment of a CM device 1 having a right side cover 51r and a left side cover 51l, according to the present invention, with the right side cover 51r and the left side cover 51l separated from the remaining parts of the CM device 1.

FIG. 17c is a front view schematic illustration of a second embodiment of a CM device 1 having right side cover and left side cover, according to the present invention. As shown by the illustration, the right side cover bottom external edge 51rb has no sharp corners, and its shape, in this view, can be slightly arched. This also applies to the left side cover bottom external edge 51lb.

FIG. 17d is a top view schematic illustration of a second embodiment of a CM device 1 having a right side cover 51r and a left side cover 51l, according to the present invention.

The right side cover 51r is connected to the body right half 10rh by means of pins (not shown in the present illustration), or any other suitable mechanical means. The left side cover 51l can be connected to the body left hand 10lh in the same manner.

Even though the present illustration shows the right side cover 51r and the left side cover 51l as mounted to the second embodiment of a CM device, the present invention is in no way limited, and they can likewise be mounted to other embodiments of the CM device.

After having described the structure and features of the CM device (1), following is a description of the method for its use according to stages of action, with the numbering of elements in this description corresponding with the numbering given in the legend of the numbering of the application illustrations that has served in illustrations of the present application and their accompanying descriptions.

The method is for cocking a CM device (1) for creating a first skin incision in open surgical procedures and simultaneously marking along the margins of the incision, the method including all or some of the following stages (not necessarily in the following order):

(a) Opening a sterile case (30) or bag and submitting a CM device (1) to a surgeon, (case (30) may be opened in advance and CM device (1) put on a sterile tray);

(b) Guarding the CM device (1) by means of a safety button (20);

(c) Holding the CM device (1) at a body upper segment (10u) of a body (10) of the CM device (1) and/or at a body back side (1ba) and/or at a body front side (1fr) segments;

(d) Adjusting a planned incision depth (501) by manipulating (e.g. rotating, pushing, pulling etc.) a depth regulator head (17a) and locking the depth regulator head (17a) by an elastic arm jag (16i) or depth regulator first arm (17g) cogs (17e) and blade holder cogs (16k);

(e) Cocking the CM device (1) by sliding a blade holder (16) against the force of an energy accumulator (21) and placing two blade holder second pins (16g) one at a body left hand tenon (10lhc) and a second at a body right hand tenon (10rhc) according to a planned incision length (502), pushing down the marker bases (11l, 11r), the stamp units (11b) and the markers (11a) into the sealing cover cells (18c), loading the markers with paint (23). In some embodiments, having a right side cover 51r and a left side cover 51l, those covers should be removed prior to cocking the CM device (1);

(f) Placing a stopper (14) at a place according to a planned incision length (502);

(g) With the surgeon's second hand or with the help of an assistant, removing a sealing cover (18) from a bottom of the body (10);

(h) Placing the CM device (1) on the skin of a patient at a selected location and orientation and, if required, pushing aside any interfering or blocking organs or flattening the skin with the hand that is not holding the CM device (1) or with help from an assistant and, when required to perform the incision on an existing outline of a scar, a wrinkle in the skin, a pre-marked line etc., then the slot at the body (10) bottom side (1bo) will be placed and centered over the scar, wrinkle, pre-marked line etc., looking on to the skin surface through the opening in the body (10). In some embodiments, having a right side cover 51r and a left side cover 51l, those covers should be reinstalled prior to placing the CM device (1) on a skin of a patient;

(i) Pressing the CM device (1) against the patient's body and flattening the patient's skin under it for at least the planed length of incision shown by painted marking on the sides of the CM device (1);

(j) Pushing sideway the safety button (20);

(k) Pushing a trigger (19), projecting a blade (15) from the CM device (1) lower surface of bottom side (1bo) to a depth required for the performance of the planned incision depth (501), moving the blade holder assembly (150), by the force of the energy accumulator (21) up to the stopper (14), thus performing a skin incision of a planned incision depth (501) and a planned incision length (502), and simultaneously marking two substantially parallel rows of colored dots one row at each side of the incision;

(l) Removing the CM device (1) from the patient;

(m) Deepening the incision to a required depth by the surgeon, using other tools and performing the rest of the surgical procedure; and

(n) Closing the incision according to the colored marks. 15

It will be noted that the method is in no way limited to include all of the aforementioned stages, may include additional stages, or may include a different order of stages.

Note that the CM device 1 can be manufactured to serve for use as a disposable, one-time-use device, or as a reusable device. Likewise, specific components can be made of transparent materials to facilitate surgeons' use of the device.

While the invention has been described with respect to a limited number of embodiments, it will be appreciated that many variations, modifications and other applications of the invention may be made.

What is claimed is:

1. A device for producing a skin incision (CM device) (1) comprising:
(a) a body (10);
(b) a blade holder assembly (150) located mostly inside said body (10);
(c) an energy accumulator (21) for moving said blade holder assembly (150), said energy accumulator (21) being located inside said body (10); and
(d) a triggering and locking assembly (190) located mostly inside said body (10).

2. The CM device (1) of claim 1 further comprising:
(e) a marking and sealing assembly (18d), attached to said body (10).

3. The CM device (1) of claim 1, wherein said body (10) has a back side (1ba), a front side (1fr), a right side (1ri), a left side (1le), a bottom side (1bo), and a body upper segment (10u), wherein said body (10) includes:
a body right half (10rh) having a body right hand bottom surface (10rha) and a polygon form around an internal polygon (1t) wherein said body right half (10rh) includes:
a body right hand track (10rhb) located at a polygon bottom side (1tb) of said body right half (10rh);
at least one body right hand tenon (10rhc) located at a polygon bottom side (1tb) of said body right half (10rh) and connected to said body right hand track (10rhb);
at least one body right hand stopper hole (10rhd) located at a polygon bottom side (1tb) of said body right half (10rh);
a body right hand pusher hole (10rhe) located at a polygon bottom side (1tb) of said body right half (10rh);
a body right hand trigger hole (10rhh) located at a polygon upper side (1tu) of said body right half (10rh); and
a body right hand safety button hole (10rhi) located at a polygon rear side (1tr) of said body right half (10rh); and
(ii) a body left half (10lh) attached to said body right half (10rh), having a body left hand bottom surface (10lha) and a polygon form around an internal polygon (1t) wherein said body left half (10lh) includes:
a body left hand track (10lhb) located at a polygon bottom side (1tb) of said body left half (10lh);
at least one body left hand tenon (10lhc) located at a polygon bottom side (1tb) of said body left half (10lh) and connected to said body left hand track (10lhb);
at least one body left hand stopper hole (10lhd) located at a polygon bottom side (1tb) of said body left half (10lh);
a body left hand pusher hole (10lhe) located at a polygon bottom side (1tb) of said body left half (10lh);
a body left hand trigger hole (10lhh) located at a polygon upper side (1tu) of said body left half (10lh); and
a body left hand safety button hole (10lhi) located at a polygon rear side (1tr) of said body left half (10lh).

4. The CM device (1) of claim 3, wherein said at least one body right hand stopper hole (10rhd) has substantially a rectangular shape, wherein said body right hand stopper hole (10rhd) is connected to said body right hand track (10rhb), wherein said body right half (10rh) further includes a body right hand trigger side hole (10rhj) located at a polygon rear side (1tr) of said body right half (10rh), wherein said at least one body left hand stopper hole (10lhd) has substantially a rectangular shape, wherein said body left hand stopper hole (10lhd) is connected to said body left hand track (10lhb), wherein said body left half (10lh) further includes a body left hand trigger side hole (10lhj) located at a polygon rear side (1tr) of said body left half (10lh).

5. The CM device (1) of claim 1 wherein said blade holder assembly (150) includes:
(i) a blade holder (16) having a blade holder base (16a), said blade holder base (16a) has a blade holder base bottom surface (16aa);
(ii) a depth regulator (17) mounted on said blade holder (16); and
(iii) a blade (15) having a blade sharp edge (15a) and blade tip (15aa), said blade (15) being connected to said depth regulator (17), wherein said blade holder (16) and said depth regulator (17) are adapted to move said blade (15) for the purpose of producing a skin incision and for controlling a blade tip projection distance (15f), of said blade tip (15aa) from said blade holder base (16a).

6. The CM device (1) of claim 1 wherein there is an angle Beta between a blade holder bottom line (16q) and a blade holder cylinder front line (16r), wherein said angle beta is substantially ninety degrees.

7. The CM device (1) of claim 1 wherein there is an angle Beta between a blade holder bottom line (16q) and a blade holder cylinder front line (16r), wherein said angle beta is substantially less than ninety degrees.

8. The CM device (1) of claim 5, wherein said blade holder (16) further includes: a blade holder cylinder (16b) having a blade holder cogs (16k) and a blade holder projection (16n), said blade holder cylinder (16b) is attached to said blade holder base (16a);
two blade holder first pins (16f) attached to said blade holder base (16a); and
two blade holder second pins (16g) attached to said blade holder base (16a), wherein said depth regulator (17) includes:
a depth regulator body (17f);
a depth regulator head (17a) attached to said depth regulator body (17f);
a depth regulator first arm (17g) having depth regulator cogs (17e), wherein said depth regulator first arm (17g) is attached to said depth regulator head (17a); and
depth regulator second arm (17h), having a depth regulator second arm projection (17i), said depth regulator second arm (17h) is attached to said depth regulator head (17a).

9. The CM device (1) of claim 7, wherein said body (10) includes: a body right half (10rh) having a body right hand bottom surface (10rha) and a polygon form around an internal polygon (1t) wherein said body right half (10rh) includes:
a body right hand track (10rhb) located at a polygon bottom side (1tb) of said body right half (10rh); a body left half (10lh) attached to said body right half (10rh), having a body left hand bottom surface (10lha) and a polygon form around an internal polygon (1t) wherein said body left half (10lh) includes:
a body left hand track (10lhb) located at a polygon bottom side (1tb) of said body left half (10lh), wherein one of said blade holder first pins (16f) and one of said blade holder second pins (16g) are located inside said body right hand track (10rhb) and wherein one of said blade holder first pin (16f) and one of said blade holder second pins (16g) are located inside said body left hand track (10lhb).

10. The CM device (1) of claim 9, wherein said blade holder assembly (150) further includes:
a blade holder eye (16e) attached to said blade holder (16).

11. The CM device (1) of claim 5, wherein said triggering and locking assembly (190) includes:
(i) a pusher (12);
(ii) a trigger flexible joint (190 attached to said pusher (12); and
(iii) a trigger (19) attached to said trigger flexible joint (19f).

12. The CM device (1) of claim 11, wherein said pusher (12) includes:
(i) a pusher base (12c);
(ii) two pusher arms (12b), wherein each one of said pusher arms (12b) is attached to said pusher base (12c); and
(iii) two pusher pivots (12a), wherein each one of said two pusher pivots (12a) is attached to one of said two pusher arms (12b),
and wherein said trigger (19) includes:
(i) a trigger main arm (19d);
(ii) a trigger spring (19b) attached to said trigger main arm (19d); and
(iii) a trigger protrusion (19a) attached to said trigger main arm (19d).

13. The CM device (1) of claim 12, wherein said trigger (19) further includes:
(iv) a trigger handle (19g) attached to said trigger main arm (19d); and
(v) two trigger side arms (19h), wherein each one of said trigger side arms (19h) is attached to said trigger main arm (19d).

14. The CM device (1) of claim 13, wherein said triggering and locking assembly (190) is made as one integral piece.

15. The CM device (1) of claim 5 further comprising:
(g) a stopper (14) for limiting said movement of said blade (15), said stopper (14) is located mostly inside said body (10); and
(h) an energy accumulator (21) for moving said blade (15) located mostly inside said body (10).

16. The CM device (1) of claim 15 wherein said stopper (14) includes:
(i) a stopper main body (14g);
(ii) a stopper bottom rail (14e) attached to said stopper main body (14g);and
(iii) a stopper handle (14h) attached to said stopper main body (14g).

17. The CM device (1) of claim 15 wherein said stopper (14) includes:
(h) a stopper (14), said stopper (14) including:
(i) a stopper central part (14a), having two stopper central part tops (14at) wherein said stopper central part (14a), is made from a flexible elastic material;
(ii) two stopper side shelves (14b) wherein each one of said two stopper side shelves (14b) is attached to one of said two stopper central part tops (14at);
(iii) two stopper vertical shelves (14c) wherein each one of said two stopper vertical shelves (14c) is attached to one of said two stopper side shelves (14b); and
(iv) two stopper pins (14d) wherein each one of said two stopper pins (14d) is attached to one of said two stopper vertical shelves (14c).

18. The CM device (1) of claim 15 wherein said energy accumulator (21) is an elastic band.

19. The CM device (1) of claim 15 wherein said energy accumulator (21) is a rubber band.

20. The CM device (1) of claim 3 wherein said body left hand (10lh) further includes a body left hand roller pin (10lhf) located at a polygon front side (1tf) of said body left half (10lh).

21. The CM device (1) of claim 20 further comprising:
(f) a roller (13) having two roller rings (13a), wherein said roller (13) is mounted on said body left hand roller pin (10lhf).

22. The CM device (1) of claim 5 further comprising:
(i) a marking and sealing assembly (18d), said marking and sealing assembly (18d) includes:
(i) a left markers base (11l) located inside said body (10);
(ii) a right markers base (11r) located inside said body (10); and
(iii) at least one marker stamp unit (11b) wherein each one of said marker stamp units (11b) includes:
a marker stamp unit base (11d);
at least one dot marker (11a) attached to said marker stamp unit base (11d); and
a marker stamp unit root (11c), wherein part of said marker stamp unit roots (11c) are inserted in said left markers base (11l) and another part of said marker stamp unit roots (11c) are inserted in said right markers base (11r).

23. The CM device (1) of claim 22 further comprising:
(j) a sealing cover base (18a); and
(k) at least one sealing cover cells (18c) attached to said sealing cover base (18a) wherein inside each one of said cover cells (18c) there is a sponge (24) and paint (23), and wherein said sealing cover (18) is mounted on said at least one marker stamp units (11b).

24. The CM device (1) of claim 23, wherein said sealing cover base (18a) has a sealing cover canal (18b).

25. The CM device (1) of claim 24, wherein each one of said marker stamp units (11b) has an inverted truncated pyramid shape.

26. The CM device (1) of claim 3 wherein said blade holder assembly (150) includes:
(i) a blade holder (16) said blade holder (16) including:
a blade holder base (16a) having a blade holder base bottom surface (16aa);
a blade holder cylinder (16b) having a blade holder cylinder bottom surface (16ba), said blade holder cylinder (16b) being attached to said blade holder base (16a);
an internal thread (16c) located inside said blade holder cylinder (16b);
a blade holder slot (16d) located inside said blade holder cylinder (16b);
two blade holder first pins (16f) attached to said blade holder base (16a);
two blade holder second pins (16g) attached to said blade holder base (16a);
a blade holder elastic arm (16h) attached to said blade holder cylinder (16b);
an elastic arm jag (16i) attached to said blade holder elastic arm (16h); and
a blade holder protrusion (16j) attached to said blade holder base bottom surface (16aa); and
(ii) a depth regulator (17), said depth regulator (17) including:
a depth regulator head (17a) having at least one depth regulator groove (17b);
a depth regulator bolt (17c) attached to said depth regulator head (17a); and
a depth regulator ring (17d) attached to said depth regulator bolt (17c); and
(iii) a blade (15) having a blade sharp edge (15a) and a blade upper end (15b), said blade (15) including:
a blade niche (15c) located at said blade upper end (15b),
wherein said blade (15) is mounted on said depth regulator (17), wherein said blade (15) is located at least partially inside said blade holder slot (16d), wherein said depth regulator bolt (17c) is combined with said internal thread (16c).

27. The CM device (1) of claim 3 wherein said body left hand track (10lhb) has a body left hand track length (10lhbl), and wherein said body right hand track (10rhb) has a body right hand track length (10rhbl).

28. The CM device (1) of claim 27 wherein said body left hand track length (10lhbl) has a length of at least four centimeters, and wherein said body right hand track length (10rhbl) has a length of at least four centimeters.

29. The CM device (1) of claim 3 further comprising:
(f) a right side cover (51r) attached to said body right hand track (10rhb); and
(g) a left side cover (51l) attached to said body left hand track (10lhb).

30. The CM device (1) of claim 1, wherein said CM device (1) is configured for enabling performing an elongated incision having a length of at least four centimeters.

31. A method comprising:
holding a CM device;
guarding said CM device (1) by means of a safety button (20);
adjusting a planned incision depth (501);
cocking said CM device (1) by sliding a blade holder (16) against a force of an energy accumulator (21) and placing two blade holder second pins (16g), one at a body left hand tenon (lOlhc) and a second at a body right hand tenon (lOrhc), according to a planned incision length (502), pushing down markers (10a) into sealing cover cells (18c), and loading said markers (10a) with paint (23);
placing a stopper (14) at a place according to a planned incision length (502);
removing a sealing cover (18) from a bottom of said CM device (1); and
pushing sideway the safety button (20).

32. The method of claim 31 further comprising:
placing said CM device (1) on the skin of a patient at a selected location and orientation;
pushing aside any interfering organs and flattening the skin around the selected location as much as necessary;
pressing the CM device (1) against the patient's body and flattening the patient's skin under said CM device (1);
pushing a trigger (19), projecting partially a blade (15) from said CM device (1) to said planned incision depth (501), moving a blade holder assembly (150), by force of said energy accumulator (21) up to said stopper (14), and performing a skin incision of said planned incision depth (501) and a planned incision length (502), and simultaneously marking two parallel rows of colored dots one row at each side of the incision parallel to it;
removing said CM device (1) from the patient;
deepening the incision to a required depth by the surgeon, using other tools and performing the rest of a surgical procedure; and
closing the incision according to the colored marks.

33. The method of claim 32 further comprising:
removing a right side cover (51r) and a left side cover (51l) prior to cocking said CM device and reinstalling said right side cover and said left side cover prior to placing said CM device on a skin of a patient.
